SEQUENCE LISTING (502 sequences)

SEQ ID NO: 1 - protein, 9 aa, Homo sapiens
YMNGTMSQV

SEQ ID NO: 2 - protein, 9 aa, Homo sapiens
MLLAVLYVL

SEQ ID NO: 3 - protein, 9 aa, Homo sapiens
YMDGTMSQV

SEQ ID NO: 4 - protein, 9 aa, Homo sapiens
MSLQRQFLR

SEQ ID NO: 5 - protein, 9 aa, Homo sapiens
SVYDFFVWL

SEQ ID NO: 6 - protein, 9 aa, Homo sapiens
LLGPGRPYR

SEQ ID NO: 7 - protein, 9 aa, Homo sapiens
SLDDYNHLV

SEQ ID NO: 8 - protein, 9 aa, Homo sapiens
TLDSQVMSL

SEQ ID NO: 9 - protein, 9 aa, Homo sapiens
VMGTLVALV

SEQ ID NO: 10 - protein, 9 aa, Homo sapiens
ITDQVPFSV

SEQ ID NO: 11 - protein, 10 aa, Homo sapiens
TITDQVPFSV

SEQ ID NO: 12 - protein, 9 aa, Homo sapiens
YLEPGVTVA

SEQ ID NO: 13 - protein, 9 aa, Homo sapiens
YLEPGVTVA

SEQ ID NO: 14 - protein, 9 aa, Homo sapiens
KTWGQYWQV

SEQ ID NO: 15 - protein, 9 aa, Homo sapiens
TWGQYWQVL

SEQ ID NO: 16 - protein, 9 aa, Homo sapiens
VLKRCLLHL

SEQ ID NO: 17 - protein, 9 aa, Homo sapiens
LNVSLADTN

SEQ ID NO: 18 - protein, 10 aa, Homo sapiens
SLADTNSLAV

SEQ ID NO: 19 - protein, 10 aa, Homo sapiens
LLDGTATLRL

SEQ ID NO: 20 - protein, 10 aa, Homo sapiens
VLYRYGSFSV

SEQ ID NO: 21 - protein, 10 aa, Homo sapiens
ALDGGNKHFL

SEQ ID NO: 22 - protein, 10 aa, Homo sapiens
VLPSPACQLV

SEQ ID NO: 23 - protein, 9 aa, Homo sapiens
IMDQVPFSV

SEQ ID NO: 24 - protein, 9 aa, Homo sapiens
AAGIGILTV

SEQ ID NO: 25 - protein, 9 aa, Homo sapiens
ILTVILGVL

SEQ ID NO: 26 - protein, 10 aa, Homo sapiens
EAAGIGILTV

SEQ ID NO: 27 - protein, 10 aa, Homo sapiens
AAGIGILTVI

SEQ ID NO: 28 - protein, 9 aa, Homo sapiens
ALEAQQEAL

SEQ ID NO: 29 - protein, 9 aa, Homo sapiens
ILESLFRAV

SEQ ID NO: 30 - protein, 10 aa, Homo sapiens
SLHCKPEEAL

SEQ ID NO: 31 - protein, 10 aa, Homo sapiens
PLVLGTLEEV

SEQ ID NO: 32 - protein, 9 aa, Homo sapiens
CLGLSYDGL

SEQ ID NO: 33 - protein, 10 aa, Homo sapiens
LLKYRAREPV

SEQ ID NO: 34 - protein, 9 aa, Homo sapiens
FLWGPRALV

SEQ ID NO: 35 - protein, 9 aa, Homo sapiens
EADPTGHSY

SEQ ID NO: 36 - protein, 8 aa, m13 coliphage insert
HWDFAWPW

SEQ ID NO: 37 - protein, 8 aa, m13 coliphage insert
FWGLWPWE

SEQ ID NO: 38 - protein, 5 aa, m13 coliphage insert
QKRAA

SEQ ID NO: 39 - protein, 5 aa, m13 coliphage insert
RRRAA

SEQ ID NO: 40 - protein, 5 aa, m13 coliphage insert
KFERQ

SEQ ID NO: 41 - protein, 8 aa, m13 coliphage insert
RGYVYQGL

SEQ ID NO: 42 - protein, 7 aa, m13 coliphage insert
YTLVQPL

SEQ ID NO: 43 - protein, 7 aa, m13 coliphage insert
TPDITPK

SEQ ID NO: 44 - protein, 7 aa, m13 coliphage insert
TYPDLRY

SEQ ID NO: 45 - protein, 7 aa, m13 coliphage insert
DRTHATS

SEQ ID NO: 46 - protein, 7 aa, m13 coliphage insert
MSTTFYS

SEQ ID NO: 47 - protein, 7 aa, m13 coliphage insert
YQHAVQT

SEQ ID NO: 48 - protein, 7 aa, m13 coliphage insert
FPFSAST

SEQ ID NO: 49 - protein, 7 aa, m13 coliphage insert
SSFPPLD

SEQ ID NO: 50 - protein, 7 aa, m13 coliphage insert
MAPSPPH

SEQ ID NO: 51 - protein, 7 aa, m13 coliphage insert
SSFPDLL

SEQ ID NO: 52 - protein, 7 aa, m13 coliphage insert
HSYNRLP

SEQ ID NO: 53 - protein, 7 aa, m13 coliphage insert
HLTHSQR

SEQ ID NO: 54 - protein, 7 aa, m13 coliphage insert
QAAQSRS

SEQ ID NO: 55 - protein, 7 aa, m13 coliphage insert
FATHHIG

SEQ ID NO: 56 - protein, 7 aa, m13 coliphage insert
SMPEPLI

SEQ ID NO: 57 - protein, 7 aa, m13 coliphage insert
IPRYHLI

SEQ ID NO: 58 - protein, 7 aa, m13 coliphage insert
SAPHMTS

SEQ ID NO: 59 - protein, 7 aa, m13 coliphage insert
KAPVWAS

SEQ ID NO: 60 - protein, 7 aa, m13 coliphage insert
LPHWLLI

SEQ ID NO: 61 - protein, 7 aa, m13 coliphage insert
ASAGYQI

SEQ ID NO: 62 - protein, 7 aa, m13 coliphage insert
VTPKTGS

SEQ ID NO: 63 - protein, 7 aa, m13 coliphage insert
EHPMPVL

SEQ ID NO: 64 - protein, 7 aa, m13 coliphage insert
VSSFVTS

SEQ ID NO: 65 - protein, 7 aa, m13 coliphage insert
STHFTWP

SEQ ID NO: 66 - protein, 7 aa, m13 coliphage insert
GQWWSPD

SEQ ID NO: 67 - protein, 7 aa, m13 coliphage insert
GPPHQDS

SEQ ID NO: 68 - protein, 7 aa, m13 coliphage insert
NTLPSTI

SEQ ID NO: 69 - protein, 7 aa, m13 coliphage insert
HQPSRWV

SEQ ID NO: 70 - protein, 7 aa, m13 coliphage insert
YGNPLQP

SEQ ID NO: 71 - protein, 7 aa, m13 coliphage insert
FHWWWQP

SEQ ID NO: 72 - protein, 7 aa, m13 coliphage insert
ITLKYPL

SEQ ID NO: 73 - protein, 7 aa, m13 coliphage insert
FHWPWLF

SEQ ID NO: 74 - protein, 7 aa, m13 coliphage insert
TAQDSTG

SEQ ID NO: 75 - protein, 7 aa, m13 coliphage insert
FHWWWQP

SEQ ID NO: 76 - protein, 7 aa, m13 coliphage insert
FHWWDWW

SEQ ID NO: 77 - protein, 7 aa, m13 coliphage insert
EPFFRMQ

SEQ ID NO: 78 - protein, 7 aa, m13 coliphage insert
TWWLNYR

SEQ ID NO: 79 - protein, 7 aa, m13 coliphage insert
FHWWWQP

SEQ ID NO: 80 - protein, 7 aa, m13 coliphage insert
QPSHLRW

SEQ ID NO: 81 - protein, 7 aa, m13 coliphage insert
SPASPVY

SEQ ID NO: 82 - protein, 7 aa, m13 coliphage insert
FHWWWQP

SEQ ID NO: 83 - protein, 7 aa, m13 coliphage insert
HPSNQAS

SEQ ID NO: 84 - protein, 7 aa, m13 coliphage insert
NSAPRPV

SEQ ID NO: 85 - protein, 7 aa, m13 coliphage insert
QLWSIYP

SEQ ID NO: 86 - protein, 7 aa, m13 coliphage insert
SWPFFDL

SEQ ID NO: 87 - protein, 7 aa, m13 coliphage insert
DTTLPLH

SEQ ID NO: 88 - protein, 7 aa, m13 coliphage insert
WHWQMLW

SEQ ID NO: 89 - protein, 7 aa, m13 coliphage insert
DSFRTPV

SEQ ID NO: 90 - protein, 7 aa, m13 coliphage insert
TSPLSLL

SEQ ID NO: 91 - protein, 7 aa, m13 coliphage insert
AYNYVSD

SEQ ID NO: 92 - protein, 7 aa, m13 coliphage insert
RPLHDPM

SEQ ID NO: 93 - protein, 7 aa, m13 coliphage insert
WPSTTLF

SEQ ID NO: 94 - protein, 7 aa, m13 coliphage insert
ATLEPVR

SEQ ID NO: 95 - protein, 7 aa, m13 coliphage insert
SMTVLRP

SEQ ID NO: 96 - protein, 7 aa, m13 coliphage insert
QIGAPSW

SEQ ID NO: 97 - protein, 7 aa, m13 coliphage insert
APDLYVP

SEQ ID NO: 98 - protein, 7 aa, m13 coliphage insert
RMPPLLP

SEQ ID NO: 99 - protein, 7 aa, m13 coliphage insert
AKATPEH

SEQ ID NO: 100 - protein, 7 aa, m13 coliphage insert
TPPLRIN

SEQ ID NO: 101 - protein, 7 aa, m13 coliphage insert
LPIHAPH

SEQ ID NO: 102 - protein, 7 aa, m13 coliphage insert
DLNAYTH

SEQ ID NO: 103 - protein, 7 aa, m13 coliphage insert
VTLPNFH

SEQ ID NO: 104 - protein, 7 aa, m13 coliphage insert
NSRLPTL

SEQ ID NO: 105 - protein, 7 aa, m13 coliphage insert
YPHPSRS

SEQ ID NO: 106 - protein, 7 aa, m13 coliphage insert
GTAHFMY

SEQ ID NO: 107 - protein, 7 aa, m13 coliphage insert
YSLLPTR

SEQ ID NO: 108 - protein, 7 aa, m13 coliphage insert
LPRRTLL

SEQ ID NO: 109 - protein, 7 aa, m13 coliphage insert
TSTLLWK

SEQ ID NO: 110 - protein, 7 aa, m13 coliphage insert
TSDMKPH

SEQ ID NO: 111 - protein, 7 aa, m13 coliphage insert
TSSYLAL

SEQ ID NO: 112 - protein, 7 aa, m13 coliphage insert
NLYGPHD

SEQ ID NO: 113 - protein, 7 aa, m13 coliphage insert
LETYTAS

SEQ ID NO: 114 - protein, 7 aa, m13 coliphage insert
AYKSLTQ

SEQ ID NO: 115 - protein, 7 aa, m13 coliphage insert
STSVYSS

SEQ ID NO: 116 - protein, 7 aa, m13 coliphage insert
EGPLRSP

SEQ ID NO: 117 - protein, 7 aa, m13 coliphage insert
TTYHALG

SEQ ID NO: 118 - protein, 7 aa, m13 coliphage insert
VSIGHPS

SEQ ID NO: 119 - protein, 7 aa, m13 coliphage insert
THSHRPS

SEQ ID NO: 120 - protein, 7 aa, m13 coliphage insert
ITNPLTT

SEQ ID NO: 121 - protein, 7 aa, m13 coliphage insert
SIQAHHS

SEQ ID NO: 122 - protein, 7 aa, m13 coliphage insert
LNWPRVL

SEQ ID NO: 123 - protein, 7 aa, m13 coliphage insert
YYYAPPP

SEQ ID NO: 124 - protein, 7 aa, m13 coliphage insert
SLWTRLP

SEQ ID NO: 125 - protein, 7 aa, m13 coliphage insert
NVYHSSL

SEQ ID NO: 126 - protein, 7 aa, m13 coliphage insert
NSPHPPT

SEQ ID NO: 127 - protein, 7 aa, m13 coliphage insert
VPAKPRH

SEQ ID NO: 128 - protein, 7 aa, m13 coliphage insert
HNLHPNR

SEQ ID NO: 129 - protein, 7 aa, m13 coliphage insert
YTTHRWL

SEQ ID NO: 130 - protein, 7 aa, m13 coliphage insert
AVTAAIV

SEQ ID NO: 131 - protein, 7 aa, m13 coliphage insert
TLMHDRV

SEQ ID NO: 132 - protein, 7 aa, m13 coliphage insert
TPLKVPY

SEQ ID NO: 133 - protein, 7 aa, m13 coliphage insert
FTNQQYH

SEQ ID NO: 134 - protein, 7 aa, m13 coliphage insert
SHVPSMA

SEQ ID NO: 135 - protein, 7 aa, m13 coliphage insert
HTTVYGA

SEQ ID NO: 136 - protein, 7 aa, m13 coliphage insert
TETPYPT

SEQ ID NO: 137 - protein, 7 aa, m13 coliphage insert
LTTPFSS

SEQ ID NO: 138 - protein, 7 aa, m13 coliphage insert
GVPLTMD

SEQ ID NO: 139 - protein, 7 aa, m13 coliphage insert
KLPTVLR

SEQ ID NO: 140 - protein, 7 aa, m13 coliphage insert
CRFHGNR

SEQ ID NO: 141 - protein, 7 aa, m13 coliphage insert
YTRDFEA

SEQ ID NO: 142 - protein, 7 aa, m13 coliphage insert
SSAAGPR

SEQ ID NO: 143 - protein, 7 aa, m13 coliphage insert
SLIQYSR

SEQ ID NO: 144 - protein, 7 aa, m13 coliphage insert
  VARIANT (7)...(7): Any amino acid residue
DALMWPX

SEQ ID NO: 145 - protein, 7 aa, m13 coliphage insert
  VARIANT (3)...(3): Any amino acid residue
SSXSLYI

SEQ ID NO: 146 - protein, 7 aa, m13 coliphage insert
FNTSTRT

SEQ ID NO: 147 - protein, 7 aa, m13 coliphage insert
TVQHVAF

SEQ ID NO: 148 - protein, 7 aa, m13 coliphage insert
DYSFPPL

SEQ ID NO: 149 - protein, 7 aa, m13 coliphage insert
VGSMESL

SEQ ID NO: 150 - protein, 7 aa, m13 coliphage insert
  VARIANT (2)...(2): Any amino acid residue
  VARIANT (6)...(6): Any amino acid residue
FXPMIXS

SEQ ID NO: 151 - protein, 7 aa, m13 coliphage insert
APPRVTM

SEQ ID NO: 152 - protein, 7 aa, m13 coliphage insert
IATKTPK

SEQ ID NO: 153 - protein, 7 aa, m13 coliphage insert
KPPLFQI

SEQ ID NO: 154 - protein, 7 aa, m13 coliphage insert
YHTAHNM

SEQ ID NO: 155 - protein, 7 aa, m13 coliphage insert
SYIQATH

SEQ ID NO: 156 - protein, 7 aa, m13 coliphage insert
SSFATFL

SEQ ID NO: 157 - protein, 7 aa, m13 coliphage insert
TTPPNFA

SEQ ID NO: 158 - protein, 7 aa, m13 coliphage insert
ISLDPRM

SEQ ID NO: 159 - protein, 7 aa, m13 coliphage insert
SLPLFGA

SEQ ID NO: 160 - protein, 7 aa, m13 coliphage insert
NLLKTTL

SEQ ID NO: 161 - protein, 7 aa, m13 coliphage insert
DQNLPRR

SEQ ID NO: 162 - protein, 7 aa, m13 coliphage insert
SHFEQLL

SEQ ID NO: 163 - protein, 7 aa, m13 coliphage insert
TPQLHHG

SEQ ID NO: 164 - protein, 7 aa, m13 coliphage insert
APLDRIT

SEQ ID NO: 165 - protein, 7 aa, m13 coliphage insert
FAPLIAH

SEQ ID NO: 166 - protein, 7 aa, m13 coliphage insert
SWIQTFM

SEQ ID NO: 167 - protein, 7 aa, m13 coliphage insert
NTWPHMY

SEQ ID NO: 168 - protein, 7 aa, m13 coliphage insert
EPLPTTL

SEQ ID NO: 169 - protein, 7 aa, m13 coliphage insert
HGPHLFN

SEQ ID NO: 170 - protein, 7 aa, m13 coliphage insert
YLNSTLA

SEQ ID NO: 171 - protein, 7 aa, m13 coliphage insert
HLHSPSG

SEQ ID NO: 172 - protein, 7 aa, m13 coliphage insert
TLPHRLN

SEQ ID NO: 173 - protein, 7 aa, m13 coliphage insert
SSPREVH

SEQ ID NO: 174 - protein, 7 aa, m13 coliphage insert
NQVDTAR

SEQ ID NO: 175 - protein, 7 aa, m13 coliphage insert
YPTPLLT

SEQ ID NO: 176 - protein, 7 aa, m13 coliphage insert
HPAAFPW

SEQ ID NO: 177 - protein, 7 aa, m13 coliphage insert
LLPHSSA

SEQ ID NO: 178 - protein, 7 aa, m13 coliphage insert
LETYTAS

SEQ ID NO: 179 - protein, 7 aa, m13 coliphage insert
KYVPLPP

SEQ ID NO: 180 - protein, 7 aa, m13 coliphage insert
APLALHA

SEQ ID NO: 181 - protein, 7 aa, m13 coliphage insert
YESLLTK

SEQ ID NO: 182 - protein, 7 aa, m13 coliphage insert
SHAASGT

SEQ ID NO: 183 - protein, 7 aa, m13 coliphage insert
GLATVKS

SEQ ID NO: 184 - protein, 7 aa, m13 coliphage insert
GATSFGL

SEQ ID NO: 185 - protein, 7 aa, m13 coliphage insert
KPPGPVS

SEQ ID NO: 186 - protein, 7 aa, m13 coliphage insert
TLYVSGN

SEQ ID NO: 187 - protein, 7 aa, m13 coliphage insert
HAPFKSQ

SEQ ID NO: 188 - protein, 7 aa, m13 coliphage insert
VAFTRLP

SEQ ID NO: 189 - protein, 7 aa, m13 coliphage insert
LPTRTPA

SEQ ID NO: 190 - protein, 7 aa, m13 coliphage insert
ASFDLLI

SEQ ID NO: 191 - protein, 7 aa, m13 coliphage insert
RMNTEPP

SEQ ID NO: 192 - protein, 7 aa, m13 coliphage insert
KMTPLTT

SEQ ID NO: 193 - protein, 7 aa, m13 coliphage insert
ANATPLL

SEQ ID NO: 194 - protein, 7 aa, m13 coliphage insert
TIWPPPV

SEQ ID NO: 195 - protein, 7 aa, m13 coliphage insert
QTKVMTT

SEQ ID NO: 196 - protein, 7 aa, m13 coliphage insert
NHAVFAS

SEQ ID NO: 197 - protein, 7 aa, m13 coliphage insert
  VARIANT (5)...(5): Any amino acid residue
LHAAXTS

SEQ ID NO: 198 - protein, 7 aa, m13 coliphage insert
TWQPYFH

SEQ ID NO: 199 - protein, 7 aa, m13 coliphage insert
APLALHA

SEQ ID NO: 200 - protein, 7 aa, m13 coliphage insert
TAHDLTV

SEQ ID NO: 201 - protein, 7 aa, m13 coliphage insert
NMTNMLT

SEQ ID NO: 202 - protein, 7 aa, m13 coliphage insert
GSGLSQD

SEQ ID NO: 203 - protein, 7 aa, m13 coliphage insert
TPIKTIY

SEQ ID NO: 204 - protein, 7 aa, m13 coliphage insert
SHLYRSS

SEQ ID NO: 205 - protein, 7 aa, m13 coliphage insert
HGQAWQF

SEQ ID NO: 206 - protein, 4 aa, m13 coliphage insert
CGSG

SEQ ID NO: 207 - protein, 9 aa, Homo sapiens derivative
YMDGTMSQV

SEQ ID NO: 208 - protein, 17 aa, Homo sapiens derivative
HWDFAWPWYMDGTMSQV

SEQ ID NO: 209 - protein, 17 aa, Homo sapiens derivative
YMDGTMSQVHWDFAWPW

SEQ ID NO: 210 - protein, 25 aa, Homo sapiens derivative
HWDFAWPWYMDGTMSQVHWDFAWPW

SEQ ID NO: 211 - protein, 25 aa, Homo sapiens derivative
HWDFAWPWYMDGTMSQVWPWAFDWH

SEQ ID NO: 212 - protein, 20 aa, Homo sapiens derivative
HWDFAWPWGSGYMDGTMSQV

SEQ ID NO: 213 - protein, 31 aa, Homo sapiens derivative
HWDFAWPWGSGYMDGTMSQVGSGWPWAFDWH

SEQ ID NO: 214 - protein, 20 aa, Homo sapiens derivative
  VARIANT (9)...(11): Any amino acid residue
HWDFAWPWXXXYMDGTMSQV

SEQ ID NO: 215 - protein, 20 aa, Homo sapiens derivative
YMDGTMSQVGSGHWDFAWPW

SEQ ID NO: 216 - protein, 31 aa, Homo sapiens derivative
  VARIANT (9)...(11): Any amino acid residue
  VARIANT (21)...(23): Any amino acid residue
HWDFAWPWXXXYMDGTMSQVXXXHWDFAWPW

SEQ ID NO: 217 - protein, 17 aa, Homo sapiens derivative
HWDFAWPWYMDGTMSQV

SEQ ID NO: 218 - protein, 17 aa, Homo sapiens derivative
YMDGTMSQVHWDFAWPW

SEQ ID NO: 219 - protein, 25 aa, Homo sapiens derivative
HWDFAWPWYMDGTMSQVHWDFAWPW

SEQ ID NO: 220 - protein, 20 aa, Homo sapiens derivative
  VARIANT (10)...(12): Any amino acid residue
YMDGTMSQVXXXWPWAFDWH

SEQ ID NO: 221 - protein, 31 aa, Homo sapiens derivative
  VARIANT (9)...(11): Any amino acid residue
  VARIANT (21)...(23): Any amino acid residue
HWDFAWPWXXXYMDGTMSQVXXXWPWAFDWH

SEQ ID NO: 222 - protein, 17 aa, Homo sapiens derivative
YMDGTMSQVWPWAFDWH

SEQ ID NO: 223 - protein, 25 aa, Homo sapiens derivative
HWDFAWPWYMDGTMSQVWPWAFDWH

SEQ ID NO: 224 - protein, 20 aa, Homo sapiens derivative
  VARIANT (9)...(11): Any amino acid residue
HWDFAWPWXXXYMNGTMSQV

SEQ ID NO: 225 - protein, 20 aa, Homo sapiens derivative
  VARIANT (10)...(12): Any amino acid residue
YMNGTMSQVXXXHWDFAWPW

SEQ ID NO: 226 - protein, 31 aa, Homo sapiens derivative
  VARIANT (9)...(11): Any amino acid residue
  VARIANT (21)...(23): Any amino acid residue
HWDFAWPWXXXYMNGTMSQVXXXHWDFAWPW

SEQ ID NO: 227 - protein, 17 aa, Homo sapiens derivative
HWDFAWPWYMNGTMSQV

SEQ ID NO: 228 - protein, 17 aa, Homo sapiens derivative
YMNGTMSQVHWDFAWPW

SEQ ID NO: 229 - protein, 25 aa, Homo sapiens derivative
HWDFAWPWYMNGTMSQVHWDFAWPW

SEQ ID NO: 230 - protein, 20 aa, Homo sapiens derivative
  VARIANT (10)...(12): Any amino acid residue
YMNGTMSQVXXXWPWAFDWH

SEQ ID NO: 231 - protein, 31 aa, Homo sapiens derivative
  VARIANT (9)...(11): Any amino acid residue
  VARIANT (21)...(23): Any amino acid residue
HWDFAWPWXXXYMNGTMSQVXXXWPWAFDWH

SEQ ID NO: 232 - protein, 17 aa, Homo sapiens derivative
YMNGTMSQVWPWAFDWH

SEQ ID NO: 233 - protein, 25 aa, Homo sapiens derivative
HWDFAWPWYMNGTMSQVWPWAFDWH

SEQ ID NO: 234 - protein, 20 aa, Homo sapiens derivative
  VARIANT (9)...(11): Any amino acid residue
HWDFAWPWXXXMLLAVLYCL

SEQ ID NO: 235 - protein, 20 aa, Homo sapiens derivative
  VARIANT (10)...(12): Any amino acid residue
MLLAVLYCLXXXHWDFAWPW

SEQ ID NO: 236 - protein, 31 aa, Homo sapiens derivative
  VARIANT (9)...(11): Any amino acid residue
  VARIANT (21)...(23): Any amino acid residue
HWDFAWPWXXXMLLAVLYCLXXXHWDFAWPW

SEQ ID NO: 237 - protein, 17 aa, Homo sapiens derivative
HWDFAWPWMLLAVLYCL

SEQ ID NO: 238 - protein, 17 aa, Homo sapiens derivative
MLLAVLYCLHWDFAWPW

SEQ ID NO: 239 - protein, 25 aa, Homo sapiens derivative
HWDFAWPWMLLAVLYCLHWDFAWPW

SEQ ID NO: 240 - protein, 20 aa, Homo sapiens derivative
  VARIANT (10)...(12): Any amino acid residue
MLLAVLYCLXXXWPWAFDWH

SEQ ID NO: 241 - protein, 31 aa, Homo sapiens derivative
  VARIANT (9)...(11): Any amino acid residue
  VARIANT (21)...(23): Any amino acid residue
HWDFAWPWXXXMLLAVLYCLXXXWPWAFDWH

SEQ ID NO: 242 - protein, 17 aa, Homo sapiens derivative
MLLAVLYCLWPWAFDWH

SEQ ID NO: 243 - protein, 25 aa, Homo sapiens derivative
HWDFAWPWMLLAVLYCLWPWAFDWH

SEQ ID NO: 244 - protein, 20 aa, Homo sapiens derivative
  VARIANT (9)...(11): Any amino acid residue
HWDFAWPWXXXIMDQVPFSV

SEQ ID NO: 245 - protein, 20 aa, Homo sapiens derivative
IMDQVPFSVGSGHWDFAWPW

SEQ ID NO: 246 - protein, 31 aa, Homo sapiens derivative
  VARIANT (9)...(11): Any amino acid residue
  VARIANT (21)...(23): Any amino acid residue
HWDFAWPWXXXIMDQVPFSVXXXHWDFAWPW

SEQ ID NO: 247 - protein, 17 aa, Homo sapiens derivative
HWDFAWPWIMDQVPFSV

SEQ ID NO: 248 - protein, 17 aa, Homo sapiens derivative
IMDQVPFSVHWDFAWPW

SEQ ID NO: 249 - protein, 25 aa, Homo sapiens derivative
HWDFAWPWIMDQVPFSVHWDFAWPW

SEQ ID NO: 250 - protein, 20 aa, Homo sapiens derivative
  VARIANT (10)...(12): Any amino acid residue
IMDQVPFSVXXXWPWAFDWH

SEQ ID NO: 251 - protein, 31 aa, Homo sapiens derivative
  VARIANT (9)...(11): Any amino acid residue
  VARIANT (21)...(23): Any amino acid residue
HWDFAWPWXXXIMDQVPFSVXXXWPWAFDWH

SEQ ID NO: 252 - protein, 17 aa, Homo sapiens derivative
IMDQVPFSVWPWAFDWH

SEQ ID NO: 253 - protein, 25 aa, Homo sapiens derivative
HWDFAWPWIMDQVPFSVWPWAFDWH

SEQ ID NO: 254 - protein, 20 aa, Homo sapiens derivative
  VARIANT (9)...(11): Any amino acid residue
HWDFAWPWXXXITDQVPFSV

SEQ ID NO: 255 - protein, 20 aa, Homo sapiens derivative
  VARIANT (10)...(12): Any amino acid residue
ITDQVPFSVXXXHWDFAWPW

SEQ ID NO: 256 - protein, 31 aa, Homo sapiens derivative
  VARIANT (9)...(11): Any amino acid residue
  VARIANT (21)...(23): Any amino acid residue
HWDFAWPWXXXITDQVPFSVXXXHWDFAWPW

SEQ ID NO: 257 - protein, 17 aa, Homo sapiens derivative
HWDFAWPWITDQVPFSV

SEQ ID NO: 258 - protein, 17 aa, Homo sapiens derivative
ITDQVPFSVHWDFAWPW

SEQ ID NO: 259 - protein, 25 aa, Homo sapiens derivative
HWDFAWPWITDQVPFSVHWDFAWPW

SEQ ID NO: 260 - protein, 20 aa, Homo sapiens derivative
  VARIANT (10)...(12): Any amino acid residue
ITDQVPFSVXXXWPWAFDWH

SEQ ID NO: 261 - protein, 31 aa, Homo sapiens derivative
  VARIANT (9)...(11): Any amino acid residue
  VARIANT (21)...(23): Any amino acid residue
HWDFAWPWXXXITDQVPFSVXXXWPWAFDWH

SEQ ID NO: 262 - protein, 17 aa, Homo sapiens derivative
ITDQVPFSVWPWAFDWH

SEQ ID NO: 263 - protein, 25 aa, Homo sapiens derivative
HWDFAWPWITDQVPFSVWPWAFDWH

SEQ ID NO: 264 - protein, 21 aa, Homo sapiens derivative
  VARIANT (9)...(11): Any amino acid residue
HWDFAWPWXXXTITDQVPFSV

SEQ ID NO: 265 - protein, 21 aa, Homo sapiens derivative
  VARIANT (11)...(13): Any amino acid residue
TITDQVPFSVXXXHWDFAWPW

SEQ ID NO: 266 - protein, 32 aa, Homo sapiens derivative
  VARIANT (9)...(11): Any amino acid residue
  VARIANT (22)...(24): Any amino acid residue
HWDFAWPWXXXTITDQVPFSVXXXHWDFAWPW

SEQ ID NO: 267 - protein, 18 aa, Homo sapiens derivative
HWDFAWPWTITDQVPFSV

SEQ ID NO: 268 - protein, 18 aa, Homo sapiens derivative
TITDQVPFSVHWDFAWPW

SEQ ID NO: 269 - protein, 26 aa, Homo sapiens derivative
HWDFAWPWTITDQVPFSVHWDFAWPW

SEQ ID NO: 270 - protein, 21 aa, Homo sapiens derivative
  VARIANT (11)...(13): Any amino acid residue
TITDQVPFSVXXXWPWAFDWH

SEQ ID NO: 271 - protein, 32 aa, Homo sapiens derivative
  VARIANT (9)...(11): Any amino acid residue
  VARIANT (22)...(24): Any amino acid residue
HWDFAWPWXXXTITDQVPFSVXXXWPWAFDWH

SEQ ID NO: 272 - protein, 18 aa, Homo sapiens derivative
TITDQVPFSVWPWAFDWH

SEQ ID NO: 273 - protein, 26 aa, Homo sapiens derivative
HWDFAWPWTITDQVPFSVWPWAFDWH

SEQ ID NO: 274 - protein, 19 aa, Homo sapiens derivative
  VARIANT (9)...(11): Any amino acid residue
HWDFAWPWXXXYLEPGVTV

SEQ ID NO: 275 - protein, 19 aa, Homo sapiens derivative
  VARIANT (9)...(11): Any amino acid residue
YLEPGVTVXXXHWDFAWPW

SEQ ID NO: 276 - protein, 30 aa, Homo sapiens derivative
  VARIANT (9)...(11): Any amino acid residue
  VARIANT (20)...(22): Any amino acid residue
HWDFAWPWXXXYLEPGVTVXXXHWDFAWPW

SEQ ID NO: 277 - protein, 16 aa, Homo sapiens derivative
HWDFAWPWYLEPGVTV

SEQ ID NO: 278 - protein, 16 aa, Homo sapiens derivative
YLEPGVTVHWDFAWPW

SEQ ID NO: 279 - protein, 24 aa, Homo sapiens derivative
HWDFAWPWYLEPGVTVHWDFAWPW

SEQ ID NO: 280 - protein, 19 aa, Homo sapiens derivative
  VARIANT (9)...(11): Any amino acid residue
YLEPGVTVXXXWPWAFDWH

SEQ ID NO: 281 - protein, 30 aa, Homo sapiens derivative
  VARIANT (9)...(11): Any amino acid residue
  VARIANT (20)...(22): Any amino acid residue
HWDFAWPWXXXYLEPGVTVXXXWPWAFDWH

SEQ ID NO: 282 - protein, 16 aa, Homo sapiens derivative
YLEPGVTVWPWAFDWH

SEQ ID NO: 283 - protein, 24 aa, Homo sapiens derivative
HWDFAWPWYLEPGVTVWPWAFDWH

SEQ ID NO: 284 - protein, 20 aa, Homo sapiens derivative
  VARIANT (9)...(11): Any amino acid residue
HWDFAWPWXXXYLEPGVTVA

SEQ ID NO: 285 - protein, 20 aa, Homo sapiens derivative
  VARIANT (10)...(12): Any amino acid residue
YLEPGVTVAXXXHWDFAWPW

SEQ ID NO: 286 - protein, 31 aa, Homo sapiens derivative
  VARIANT (9)...(11): Any amino acid residue
  VARIANT (21)...(23): Any amino acid residue
HWDFAWPWXXXYLEPGVTVAXXXHWDFAWPW

SEQ ID NO: 287 - protein, 17 aa, Homo sapiens derivative
HWDFAWPWYLEPGVTVA

SEQ ID NO: 288 - protein, 17 aa, Homo sapiens derivative
YLEPGVTVAHWDFAWPW

SEQ ID NO: 289 - protein, 25 aa, Homo sapiens derivative
HWDFAWPWYLEPGVTVAHWDFAWPW

SEQ ID NO: 290 - protein, 20 aa, Homo sapiens derivative
  VARIANT (10)...(12): Any amino acid residue
YLEPGVTVAXXXWPWAFDWH

SEQ ID NO: 291 - protein, 31 aa, Homo sapiens derivative
  VARIANT (9)...(11): Any amino acid residue
  VARIANT (21)...(23): Any amino acid residue
HWDFAWPWXXXYLEPGVTVAXXXWPWAFDWH

SEQ ID NO: 292 - protein, 17 aa, Homo sapiens derivative
YLEPGVTVAWPWAFDWH

SEQ ID NO: 293 - protein, 25 aa, Homo sapiens derivative
HWDFAWPWYLEPGVTVAWPWAFDWH

SEQ ID NO: 294 - protein, 20 aa, Homo sapiens derivative
  VARIANT (9)...(11): Any amino acid residue
HWDFAWPWXXXKTWGQYWQV

SEQ ID NO: 295 - protein, 20 aa, Homo sapiens derivative
  VARIANT (10)...(12): Any amino acid residue
KTWGQYWQVXXXHWDFAWPW

SEQ ID NO: 296 - protein, 31 aa, Homo sapiens derivative
  VARIANT (9)...(11): Any amino acid residue
  VARIANT (21)...(23): Any amino acid residue
HWDFAWPWXXXKTWGQYWQVXXXHWDFAWPW

SEQ ID NO: 297 - protein, 17 aa, Homo sapiens derivative
HWDFAWPWKTWGQYWQV

SEQ ID NO: 298 - protein, 17 aa, Homo sapiens derivative
KTWGQYWQVHWDFAWPW

SEQ ID NO: 299 - protein, 25 aa, Homo sapiens derivative
HWDFAWPWKTWGQYWQVHWDFAWPW

SEQ ID NO: 300 - protein, 20 aa, Homo sapiens derivative
  VARIANT (10)...(12): Any amino acid residue
KTWGQYWQVXXXWPWAFDWH

SEQ ID NO: 301 - protein, 31 aa, Homo sapiens derivative
  VARIANT (9)...(11): Any amino acid residue
  VARIANT (21)...(23): Any amino acid residue
HWDFAWPWXXXKTWGQYWQVXXXWPWAFDWH

SEQ ID NO: 302 - protein, 17 aa, Homo sapiens derivative
KTWGQYWQVWPWAFDWH

SEQ ID NO: 303 - protein, 25 aa, Homo sapiens derivative
HWDFAWPWKTWGQYWQVWPWAFDWH

SEQ ID NO: 304 - protein, 21 aa, Homo sapiens derivative
  VARIANT (9)...(11): Any amino acid residue
HWDFAWPWXXXKTWGQYWQVL

SEQ ID NO: 305 - protein, 21 aa, Homo sapiens derivative
  VARIANT (11)...(13): Any amino acid residue
KTWGQYWQVLXXXHWDFAWPW

SEQ ID NO: 306 - protein, 32 aa, Homo sapiens derivative
  VARIANT (9)...(11): Any amino acid residue
  VARIANT (22)...(24): Any amino acid residue
HWDFAWPWXXXKTWGQYWQVLXXXHWDFAWPW

SEQ ID NO: 307 - protein, 18 aa, Homo sapiens derivative
HWDFAWPWKTWGQYWQVL

SEQ ID NO: 308 - protein, 18 aa, Homo sapiens derivative
KTWGQYWQVLHWDFAWPW

SEQ ID NO: 309 - protein, 26 aa, Homo sapiens derivative
HWDFAWPWKTWGQYWQVLHWDFAWPW

SEQ ID NO: 310 - protein, 21 aa, Homo sapiens derivative
  VARIANT (11)...(13): Any amino acid residue
KTWGQYWQVLXXXWPWAFDWH

SEQ ID NO: 311 - protein, 32 aa, Homo sapiens derivative
  VARIANT (9)...(11): Any amino acid residue
  VARIANT (22)...(24): Any amino acid residue
HWDFAWPWXXXKTWGQYWQVLXXXWPWAFDWH

SEQ ID NO: 312 - protein, 18 aa, Homo sapiens derivative
KTWGQYWQVLWPWAFDWH

SEQ ID NO: 313 - protein, 26 aa, Homo sapiens derivative
HWDFAWPWKTWGQYWQVLWPWAFDWH

SEQ ID NO: 314 - protein, 20 aa, Homo sapiens derivative
  VARIANT (9)...(11): Any amino acid residue
HWDFAWPWXXXVLKRCLLHL

SEQ ID NO: 315 - protein, 20 aa, Homo sapiens derivative
  VARIANT (10)...(12): Any amino acid residue
VLKRCLLHLXXXHWDFAWPW

SEQ ID NO: 316 - protein, 31 aa, Homo sapiens derivative
  VARIANT (9)...(11): Any amino acid residue
  VARIANT (21)...(23): Any amino acid residue
HWDFAWPWXXXVLKRCLLHLXXXHWDFAWPW

SEQ ID NO: 317 - protein, 17 aa, Homo sapiens derivative
HWDFAWPWVLKRCLLHL

SEQ ID NO: 318 - protein, 17 aa, Homo sapiens derivative
VLKRCLLHLHWDFAWPW

SEQ ID NO: 319 - protein, 25 aa, Homo sapiens derivative
HWDFAWPWVLKRCLLHLHWDFAWPW

SEQ ID NO: 320 - protein, 20 aa, Homo sapiens derivative
  VARIANT (10)...(12): Any amino acid residue
VLKRCLLHLXXXWPWAFDWH

SEQ ID NO: 321 - protein, 31 aa, Homo sapiens derivative
  VARIANT (9)...(11): Any amino acid residue
  VARIANT (21)...(23): Any amino acid residue
HWDFAWPWXXXVLKRCLLHLXXXWPWAFDWH

SEQ ID NO: 322 - protein, 17 aa, Homo sapiens derivative
VLKRCLLHLWPWAFDWH

SEQ ID NO: 323 - protein, 25 aa, Homo sapiens derivative
HWDFAWPWVLKRCLLHLWPWAFDWH

SEQ ID NO: 324 - protein, 20 aa, Homo sapiens derivative
  VARIANT (9)...(11): Any amino acid residue
HWDFAWPWXXXLNVSLADTN

SEQ ID NO: 325 - protein, 20 aa, Homo sapiens derivative
  VARIANT (10)...(12): Any amino acid residue
LNVSLADTNXXXHWDFAWPW

SEQ ID NO: 326 - protein, 31 aa, Homo sapiens derivative
  VARIANT (9)...(11): Any amino acid residue
  VARIANT (21)...(23): Any amino acid residue
HWDFAWPWXXXLNVSLADTNXXXHWDFAWPW

SEQ ID NO: 327 - protein, 17 aa, Homo sapiens derivative
HWDFAWPWLNVSLADTN

SEQ ID NO: 328 - protein, 17 aa, Homo sapiens derivative
LNVSLADTNHWDFAWPW

SEQ ID NO: 329 - protein, 25 aa, Homo sapiens derivative
HWDFAWPWLNVSLADTNHWDFAWPW

SEQ ID NO: 330 - protein, 20 aa, Homo sapiens derivative
  VARIANT (10)...(12): Any amino acid residue
LNVSLADTNXXXWPWAFDWH

SEQ ID NO: 331 - protein, 31 aa, Homo sapiens derivative
  VARIANT (9)...(11): Any amino acid residue
  VARIANT (21)...(23): Any amino acid residue
HWDFAWPWXXXLNVSLADTNXXXWPWAFDWH

SEQ ID NO: 332 - protein, 17 aa, Homo sapiens derivative
LNVSLADTNWPWAFDWH

SEQ ID NO: 333 - protein, 25 aa, Homo sapiens derivative
HWDFAWPWLNVSLADTNWPWAFDWH

SEQ ID NO: 334 - protein, 21 aa, Homo sapiens derivative
  VARIANT (9)...(11): Any amino acid residue
HWDFAWPWXXXSLADTNSLAV

SEQ ID NO: 335 - protein, 21 aa, Homo sapiens derivative
  VARIANT (11)...(13): Any amino acid residue
SLADTNSLAVXXXHWDFAWPW

SEQ ID NO: 336 - protein, 32 aa, Homo sapiens derivative
  VARIANT (9)...(11): Any amino acid residue
  VARIANT (22)...(24): Any amino acid residue
HWDFAWPWXXXSLADTNSLAVXXXHWDFAWPW

SEQ ID NO: 337 - protein, 18 aa, Homo sapiens derivative
HWDFAWPWSLADTNSLAV

SEQ ID NO: 338 - protein, 18 aa, Homo sapiens derivative
SLADTNSLAVHWDFAWPW

SEQ ID NO: 339 - protein, 26 aa, Homo sapiens derivative
HWDFAWPWSLADTNSLAVHWDFAWPW

SEQ ID NO: 340 - protein, 21 aa, Homo sapiens derivative
  VARIANT (11)...(13): Any amino acid residue
SLADTNSLAVXXXWPWAFDWH

SEQ ID NO: 341 - protein, 32 aa, Homo sapiens derivative
  VARIANT (9)...(11): Any amino acid residue
  VARIANT (22)...(24): Any amino acid residue
HWDFAWPWXXXSLADTNSLAVXXXWPWAFDWH

SEQ ID NO: 342 - protein, 18 aa, Homo sapiens derivative
SLADTNSLAVWPWAFDWH

SEQ ID NO: 343 - protein, 26 aa, Homo sapiens derivative
HWDFAWPWSLADTNSLAVWPWAFDWH

SEQ ID NO: 344 - protein, 21 aa, Homo sapiens derivative
  VARIANT (9)...(11): Any amino acid residue
HWDFAWPWXXXLLDGTATLRL

SEQ ID NO: 345 - protein, 21 aa, Homo sapiens derivative
  VARIANT (11)...(13): Any amino acid residue
LLDGTATLRLXXXHWDFAWPW

SEQ ID NO: 346 - protein, 32 aa, Homo sapiens derivative
  VARIANT (9)...(11): Any amino acid residue
  VARIANT (22)...(24): Any amino acid residue
HWDFAWPWXXXLLDGTATLRLXXXHWDFAWPW

SEQ ID NO: 347 - protein, 18 aa, Homo sapiens derivative
HWDFAWPWLLDGTATLRL

SEQ ID NO: 348 - protein, 18 aa, Homo sapiens derivative
LLDGTATLRLHWDFAWPW

SEQ ID NO: 349 - protein, 26 aa, Homo sapiens derivative
HWDFAWPWLLDGTATLRLHWDFAWPW

SEQ ID NO: 350 - protein, 21 aa, Homo sapiens derivative
  VARIANT (11)...(13): Any amino acid residue
LLDGTATLRLXXXWPWAFDWH

SEQ ID NO: 351 - protein, 32 aa, Homo sapiens derivative
  VARIANT (9)...(11): Any amino acid residue
  VARIANT (22)...(24): Any amino acid residue
HWDFAWPWXXXLLDGTATLRLXXXWPWAFDWH

SEQ ID NO: 352 - protein, 18 aa, Homo sapiens derivative
LLDGTATLRLWPWAFDWH

SEQ ID NO: 353 - protein, 26 aa, Homo sapiens derivative
HWDFAWPWLLDGTATLRLWPWAFDWH

SEQ ID NO: 354 - protein, 21 aa, Homo sapiens derivative
  VARIANT (9)...(11): Any amino acid residue
HWDFAWPWXXXVLYRYGSFSV

SEQ ID NO: 355 - protein, 21 aa, Homo sapiens derivative
  VARIANT (11)...(13): Any amino acid residue
VLYRYGSFSVXXXHWDFAWPW

SEQ ID NO: 356 - protein, 32 aa, Homo sapiens derivative
  VARIANT (9)...(11): Any amino acid residue
  VARIANT (22)...(24): Any amino acid residue
HWDFAWPWXXXVLYRYGSFSVXXXHWDFAWPW

SEQ ID NO: 357 - protein, 18 aa, Homo sapiens derivative
HWDFAWPWVLYRYGSFSV

SEQ ID NO: 358 - protein, 18 aa, Homo sapiens derivative
VLYRYGSFSVHWDFAWPW

SEQ ID NO: 359 - protein, 26 aa, Homo sapiens derivative
HWDFAWPWVLYRYGSFSVHWDFAWPW

SEQ ID NO: 360 - protein, 21 aa, Homo sapiens derivative
  VARIANT (11)...(13): Any amino acid residue
VLYRYGSFSVXXXWPWAFDWH

SEQ ID NO: 361 - protein, 32 aa, Homo sapiens derivative
  VARIANT (9)...(11): Any amino acid residue
  VARIANT (22)...(24): Any amino acid residue
HWDFAWPWXXXVLYRYGSFSVXXXWPWAFDWH

SEQ ID NO: 362 - protein, 18 aa, Homo sapiens derivative
VLYRYGSFSVWPWAFDWH

SEQ ID NO: 363 - protein, 26 aa, Homo sapiens derivative
HWDFAWPWVLYRYGSFSVWPWAFDWH

SEQ ID NO: 364 - protein, 21 aa, Homo sapiens derivative
  VARIANT (9)...(11): Any amino acid residue
HWDFAWPWXXXALDGGNKHFL

SEQ ID NO: 365 - protein, 21 aa, Homo sapiens derivative
  VARIANT (11)...(13): Any amino acid residue
ALDGGNKHFLXXXHWDFAWPW

SEQ ID NO: 366 - protein, 32 aa, Homo sapiens derivative
  VARIANT (9)...(11): Any amino acid residue
  VARIANT (22)...(24): Any amino acid residue
HWDFAWPWXXXALDGGNKHFLXXXHWDFAWPW

SEQ ID NO: 367 - protein, 18 aa, Homo sapiens derivative
HWDFAWPWALDGGNKHFL

SEQ ID NO: 368 - protein, 18 aa, Homo sapiens derivative
ALDGGNKHFLHWDFAWPW

SEQ ID NO: 369 - protein, 26 aa, Homo sapiens derivative
HWDFAWPWALDGGNKHFLHWDFAWPW

SEQ ID NO: 370 - protein, 21 aa, Homo sapiens derivative
  VARIANT (11)...(13): Any amino acid residue
ALDGGNKHFLXXXWPWAFDWH

SEQ ID NO: 371 - protein, 32 aa, Homo sapiens derivative
  VARIANT (9)...(11): Any amino acid residue
  VARIANT (22)...(24): Any amino acid residue
HWDFAWPWXXXALDGGNKHFLXXXWPWAFDWH

SEQ ID NO: 372 - protein, 18 aa, Homo sapiens derivative
ALDGGNKHFLWPWAFDWH

SEQ ID NO: 373 - protein, 26 aa, Homo sapiens derivative
HWDFAWPWALDGGNKHFLWPWAFDWH

SEQ ID NO: 374 - protein, 21 aa, Homo sapiens derivative
  VARIANT (9)...(11): Any amino acid residue
HWDFAWPWXXXVLPSPACQLV

SEQ ID NO: 375 - protein, 21 aa, Homo sapiens derivative
  VARIANT (11)...(13): Any amino acid residue
VLPSPACQLVXXXHWDFAWPW

SEQ ID NO: 376 - protein, 32 aa, Homo sapiens derivative
  VARIANT (9)...(11): Any amino acid residue
  VARIANT (22)...(24): Any amino acid residue
HWDFAWPWXXXVLPSPACQLVXXXHWDFAWPW

SEQ ID NO: 377 - protein, 18 aa, Homo sapiens derivative
HWDFAWPWVLPSPACQLV

SEQ ID NO: 378 - protein, 18 aa, Homo sapiens derivative
VLPSPACQLVHWDFAWPW

SEQ ID NO: 379 - protein, 26 aa, Homo sapiens derivative
HWDFAWPWVLPSPACQLVHWDFAWPW

SEQ ID NO: 380 - protein, 21 aa, Homo sapiens derivative
  VARIANT (11)...(13): Any amino acid residue
VLPSPACQLVXXXWPWAFDWH

SEQ ID NO: 381 - protein, 32 aa, Homo sapiens derivative
  VARIANT (9)...(11): Any amino acid residue
  VARIANT (22)...(24): Any amino acid residue
HWDFAWPWXXXVLPSPACQLVXXXWPWAFDWH

SEQ ID NO: 382 - protein, 18 aa, Homo sapiens derivative
VLPSPACQLVWPWAFDWH

SEQ ID NO: 383 - protein, 26 aa, Homo sapiens derivative
HWDFAWPWVLPSPACQLVWPWAFDWH

SEQ ID NO: 384 - protein, 20 aa, Homo sapiens derivative
  VARIANT (9)...(11): Any amino acid residue
HWDFAWPWXXXAAGIGILTV

SEQ ID NO: 385 - protein, 20 aa, Homo sapiens derivative
  VARIANT (10)...(12): Any amino acid residue
AAGIGILTVXXXHWDFAWPW

SEQ ID NO: 386 - protein, 31 aa, Homo sapiens derivative
  VARIANT (9)...(11): Any amino acid residue
  VARIANT (21)...(23): Any amino acid residue
HWDFAWPWXXXAAGIGILTVXXXHWDFAWPW

SEQ ID NO: 387 - protein, 17 aa, Homo sapiens derivative
HWDFAWPWAAGIGILTV

SEQ ID NO: 388 - protein, 17 aa, Homo sapiens derivative
AAGIGILTVHWDFAWPW

SEQ ID NO: 389 - protein, 25 aa, Homo sapiens derivative
HWDFAWPWAAGIGILTVHWDFAWPW

SEQ ID NO: 390 - protein, 20 aa, Homo sapiens derivative
  VARIANT (10)...(12): Any amino acid residue
AAGIGILTVXXXWPWAFDWH

SEQ ID NO: 391 - protein, 31 aa, Homo sapiens derivative
  VARIANT (9)...(11): Any amino acid residue
  VARIANT (21)...(23): Any amino acid residue
HWDFAWPWXXXAAGIGILTVXXXWPWAFDWH

SEQ ID NO: 392 - protein, 17 aa, Homo sapiens derivative
AAGIGILTVWPWAFDWH

SEQ ID NO: 393 - protein, 25 aa, Homo sapiens derivative
HWDFAWPWAAGIGILTVWPWAFDWH

SEQ ID NO: 394 - protein, 21 aa, Homo sapiens derivative
  VARIANT (9)...(11): Any amino acid residue
HWDFAWPWXXXEAAGIGILTV

SEQ ID NO: 395 - protein, 21 aa, Homo sapiens derivative
  VARIANT (11)...(13): Any amino acid residue
EAAGIGILTVXXXHWDFAWPW

SEQ ID NO: 396 - protein, 32 aa, Homo sapiens derivative
  VARIANT (9)...(11): Any amino acid residue
  VARIANT (22)...(24): Any amino acid residue
HWDFAWPWXXXEAAGIGILTVXXXHWDFAWPW

SEQ ID NO: 397 - protein, 18 aa, Homo sapiens derivative
HWDFAWPWEAAGIGILTV

SEQ ID NO: 398 - protein, 18 aa, Homo sapiens derivative
EAAGIGILTVHWDFAWPW

SEQ ID NO: 399 - protein, 26 aa, Homo sapiens derivative
HWDFAWPWEAAGIGILTVHWDFAWPW

SEQ ID NO: 400 - protein, 21 aa, Homo sapiens derivative
  VARIANT (11)...(13): Any amino acid residue
EAAGIGILTVXXXWPWAFDWH

SEQ ID NO: 401 - protein, 32 aa, Homo sapiens derivative
  VARIANT (9)...(11): Any amino acid residue
  VARIANT (22)...(24): Any amino acid residue
HWDFAWPWXXXEAAGIGILTVXXXWPWAFDWH

SEQ ID NO: 402 - protein, 18 aa, Homo sapiens derivative
EAAGIGILTVWPWAFDWH

SEQ ID NO: 403 - protein, 26 aa, Homo sapiens derivative
HWDFAWPWEAAGIGILTVWPWAFDWH

SEQ ID NO: 404 - protein, 21 aa, Homo sapiens derivative
  VARIANT (9)...(11): Any amino acid residue
HWDFAWPWXXXEAAGGILTVI

SEQ ID NO: 405 - protein, 21 aa, Homo sapiens derivative
  VARIANT (11)...(13): Any amino acid residue
EAAGGILTVIXXXHWDFAWPW

SEQ ID NO: 406 - protein, 32 aa, Homo sapiens derivative
  VARIANT (9)...(11): Any amino acid residue
  VARIANT (22)...(24): Any amino acid residue
HWDFAWPWXXXEAAGGILTVIXXXHWDFAWPW

SEQ ID NO: 407 - protein, 18 aa, Homo sapiens derivative
HWDFAWPWEAAGGILTVI

SEQ ID NO: 408 - protein, 18 aa, Homo sapiens derivative
EAAGGILTVIHWDFAWPW

SEQ ID NO: 409 - protein, 26 aa, Homo sapiens derivative
HWDFAWPWEAAGGILTVIHWDFAWPW

SEQ ID NO: 410 - protein, 21 aa, Homo sapiens derivative
  VARIANT (11)...(13): Any amino acid residue
EAAGGILTVIXXXWPWAFDWH

SEQ ID NO: 411 - protein, 32 aa, Homo sapiens derivative
  VARIANT (9)...(11): Any amino acid residue
  VARIANT (22)...(24): Any amino acid residue
HWDFAWPWXXXEAAGGILTVIXXXWPWAFDWH

SEQ ID NO: 412 - protein, 18 aa, Homo sapiens derivative
EAAGGILTVIWPWAFDWH

SEQ ID NO: 413 - protein, 26 aa, Homo sapiens derivative
HWDFAWPWEAAGGILTVIWPWAFDWH

SEQ ID NO: 414 - protein, 20 aa, Homo sapiens derivative
  VARIANT (9)...(11): Any amino acid residue
HWDFAWPWXXXILTVILGVL

SEQ ID NO: 415 - protein, 20 aa, Homo sapiens derivative
  VARIANT (10)...(12): Any amino acid residue
ILTVILGVLXXXHWDFAWPW

SEQ ID NO: 416 - protein, 31 aa, Homo sapiens derivative
  VARIANT (9)...(11): Any amino acid residue
  VARIANT (21)...(23): Any amino acid residue
HWDFAWPWXXXILTVILGVLXXXHWDFAWPW

SEQ ID NO: 417 - protein, 17 aa, Homo sapiens derivative
HWDFAWPWILTVILGVL

SEQ ID NO: 418 - protein, 17 aa, Homo sapiens derivative
ILTVILGVLHWDFAWPW

SEQ ID NO: 419 - protein, 25 aa, Homo sapiens derivative
HWDFAWPWILTVILGVLHWDFAWPW

SEQ ID NO: 420 - protein, 20 aa, Homo sapiens derivative
  VARIANT (10)...(12): Any amino acid residue
ILTVILGVLXXXWPWAFDWH

SEQ ID NO: 421 - protein, 31 aa, Homo sapiens derivative
  VARIANT (9)...(11): Any amino acid residue
  VARIANT (21)...(23): Any amino acid residue
HWDFAWPWXXXILTVILGVLXXXWPWAFDWH

SEQ ID NO: 422 - protein, 17 aa, Homo sapiens derivative
ILTVILGVLWPWAFDWH

SEQ ID NO: 423 - protein, 25 aa, Homo sapiens derivative
HWDFAWPWILTVILGVLWPWAFDWH

SEQ ID NO: 424 - protein, 20 aa, Homo sapiens derivative
  VARIANT (9)...(11): Any amino acid residue
HWDFAWPWXXXALEAQQEAL

SEQ ID NO: 425 - protein, 20 aa, Homo sapiens derivative
  VARIANT (10)...(12): Any amino acid residue
ALEAQQEALXXXHWDFAWPW

SEQ ID NO: 426 - protein, 31 aa, Homo sapiens derivative
  VARIANT (9)...(11): Any amino acid residue
  VARIANT (21)...(23): Any amino acid residue
HWDFAWPWXXXALEAQQEALXXXHWDFAWPW

SEQ ID NO: 427 - protein, 17 aa, Homo sapiens derivative
HWDFAWPWALEAQQEAL

SEQ ID NO: 428 - protein, 17 aa, Homo sapiens derivative
ALEAQQEALHWDFAWPW

SEQ ID NO: 429 - protein, 25 aa, Homo sapiens derivative
HWDFAWPWALEAQQEALHWDFAWPW

SEQ ID NO: 430 - protein, 20 aa, Homo sapiens derivative
  VARIANT (10)...(12): Any amino acid residue
ALEAQQEALXXXWPWAFDWH

SEQ ID NO: 431 - protein, 31 aa, Homo sapiens derivative
  VARIANT (9)...(11): Any amino acid residue
  VARIANT (21)...(23): Any amino acid residue
HWDFAWPWXXXALEAQQEALXXXWPWAFDWH

SEQ ID NO: 432 - protein, 17 aa, Homo sapiens derivative
ALEAQQEALWPWAFDWH

SEQ ID NO: 433 - protein, 25 aa, Homo sapiens derivative
HWDFAWPWALEAQQEALWPWAFDWH

SEQ ID NO: 434 - protein, 20 aa, Homo sapiens derivative
  VARIANT (9)...(11): Any amino acid residue
HWDFAWPWXXXILESLFRAV

SEQ ID NO: 435 - protein, 20 aa, Homo sapiens derivative
  VARIANT (10)...(12): Any amino acid residue
ILESLFRAVXXXHWDFAWPW

SEQ ID NO: 436 - protein, 31 aa, Homo sapiens derivative
  VARIANT (9)...(11): Any amino acid residue
  VARIANT (21)...(23): Any amino acid residue
HWDFAWPWXXXILESLFRAVXXXHWDFAWPW

SEQ ID NO: 437 - protein, 17 aa, Homo sapiens derivative
HWDFAWPWILESLFRAV

SEQ ID NO: 438 - protein, 17 aa, Homo sapiens derivative
ILESLFRAVHWDFAWPW

SEQ ID NO: 439 - protein, 25 aa, Homo sapiens derivative
HWDFAWPWILESLFRAVHWDFAWPW

SEQ ID NO: 440 - protein, 20 aa, Homo sapiens derivative
  VARIANT (10)...(12): Any amino acid residue
ILESLFRAVXXXWPWAFDWH

SEQ ID NO: 441 - protein, 31 aa, Homo sapiens derivative
  VARIANT (9)...(11): Any amino acid residue
  VARIANT (21)...(23): Any amino acid residue
HWDFAWPWXXXILESLFRAVXXXWPWAFDWH

SEQ ID NO: 442 - protein, 17 aa, Homo sapiens derivative
ILESLFRAVWPWAFDWH

SEQ ID NO: 443 - protein, 25 aa, Homo sapiens derivative
HWDFAWPWILESLFRAVWPWAFDWH

SEQ ID NO: 444 - protein, 21 aa, Homo sapiens derivative
  VARIANT (9)...(11): Any amino acid residue
HWDFAWPWXXXSLHCKPEEAL

SEQ ID NO: 445 - protein, 21 aa, Homo sapiens derivative
  VARIANT (11)...(13): Any amino acid residue
SLHCKPEEALXXXHWDFAWPW

SEQ ID NO: 446 - protein, 32 aa, Homo sapiens derivative
  VARIANT (9)...(11): Any amino acid residue
  VARIANT (22)...(24): Any amino acid residue
HWDFAWPWXXXSLHCKPEEALXXXHWDFAWPW

SEQ ID NO: 447 - protein, 18 aa, Homo sapiens derivative
HWDFAWPWSLHCKPEEAL

SEQ ID NO: 448 - protein, 18 aa, Homo sapiens derivative
SLHCKPEEALHWDFAWPW

SEQ ID NO: 449 - protein, 26 aa, Homo sapiens derivative
HWDFAWPWSLHCKPEEALHWDFAWPW

SEQ ID NO: 450 - protein, 21 aa, Homo sapiens derivative
  VARIANT (11)...(13): Any amino acid residue
SLHCKPEEALXXXWPWAFDWH

SEQ ID NO: 451 - protein, 32 aa, Homo sapiens derivative
  VARIANT (9)...(11): Any amino acid residue
  VARIANT (22)...(24): Any amino acid residue
HWDFAWPWXXXSLHCKPEEALXXXWPWAFDWH

SEQ ID NO: 452 - protein, 18 aa, Homo sapiens derivative
SLHCKPEEALWPWAFDWH

SEQ ID NO: 453 - protein, 26 aa, Homo sapiens derivative
HWDFAWPWSLHCKPEEALWPWAFDWH

SEQ ID NO: 454 - protein, 21 aa, Homo sapiens derivative
  VARIANT (9)...(11): Any amino acid residue
HWDFAWPWXXXPLVLGTLEEV

SEQ ID NO: 455 - protein, 21 aa, Homo sapiens derivative
  VARIANT (11)...(13): Any amino acid residue
PLVLGTLEEVXXXHWDFAWPW

SEQ ID NO: 456 - protein, 32 aa, Homo sapiens derivative
  VARIANT (9)...(11): Any amino acid residue
  VARIANT (22)...(24): Any amino acid residue
HWDFAWPWXXXPLVLGTLEEVXXXHWDFAWPW

SEQ ID NO: 457 - protein, 18 aa, Homo sapiens derivative
HWDFAWPWPLVLGTLEEV

SEQ ID NO: 458 - protein, 18 aa, Homo sapiens derivative
PLVLGTLEEVHWDFAWPW

SEQ ID NO: 459 - protein, 26 aa, Homo sapiens derivative
HWDFAWPWPLVLGTLEEVHWDFAWPW

SEQ ID NO: 460 - protein, 21 aa, Homo sapiens derivative
  VARIANT (11)...(13): Any amino acid residue
PLVLGTLEEVXXXWPWAFDWH

SEQ ID NO: 461 - protein, 32 aa, Homo sapiens derivative
  VARIANT (9)...(11): Any amino acid residue
  VARIANT (22)...(24): Any amino acid residue
HWDFAWPWXXXPLVLGTLEEVXXXWPWAFDWH

SEQ ID NO: 462 - protein, 18 aa, Homo sapiens derivative
PLVLGTLEEVWPWAFDWH

SEQ ID NO: 463 - protein, 27 aa, Homo sapiens derivative
HWDFAWPWIPLVLGTLEEVWPWAFDWH

SEQ ID NO: 464 - protein, 20 aa, Homo sapiens derivative
  VARIANT (9)...(11): Any amino acid residue
HWDFAWPWXXXCLGLSYDGL

SEQ ID NO: 465 - protein, 20 aa, Homo sapiens derivative
  VARIANT (10)...(12): Any amino acid residue
CLGLSYDGLXXXHWDFAWPW

SEQ ID NO: 466 - protein, 31 aa, Homo sapiens derivative
  VARIANT (9)...(11): Any amino acid residue
  VARIANT (21)...(23): Any amino acid residue
HWDFAWPWXXXCLGLSYDGLXXXHWDFAWPW

SEQ ID NO: 467 - protein, 17 aa, Homo sapiens derivative
HWDFAWPWCLGLSYDGL

SEQ ID NO: 468 - protein, 17 aa, Homo sapiens derivative
CLGLSYDGLHWDFAWPW

SEQ ID NO: 469 - protein, 25 aa, Homo sapiens derivative
HWDFAWPWCLGLSYDGLHWDFAWPW

SEQ ID NO: 470 - protein, 20 aa, Homo sapiens derivative
  VARIANT (10)...(12): Any amino acid residue
CLGLSYDGLXXXWPWAFDWH

SEQ ID NO: 471 - protein, 31 aa, Homo sapiens derivative
  VARIANT (9)...(11): Any amino acid residue
  VARIANT (21)...(23): Any amino acid residue
HWDFAWPWXXXCLGLSYDGLXXXWPWAFDWH

SEQ ID NO: 472 - protein, 17 aa, Homo sapiens derivative
CLGLSYDGLWPWAFDWH

SEQ ID NO: 473 - protein, 25 aa, Homo sapiens derivative
HWDFAWPWCLGLSYDGLWPWAFDWH

SEQ ID NO: 474 - protein, 21 aa, Homo sapiens derivative
  VARIANT (9)...(11): Any amino acid residue
HWDFAWPWXXXCLGLSYDGLL

SEQ ID NO: 475 - protein, 21 aa, Homo sapiens derivative
  VARIANT (11)...(13): Any amino acid residue
CLGLSYDGLLXXXHWDFAWPW

SEQ ID NO: 476 - protein, 32 aa, Homo sapiens derivative
  VARIANT (9)...(11): Any amino acid residue
  VARIANT (22)...(24): Any amino acid residue
HWDFAWPWXXXCLGLSYDGLLXXXHWDFAWPW

SEQ ID NO: 477 - protein, 18 aa, Homo sapiens derivative
HWDFAWPWCLGLSYDGLL

SEQ ID NO: 478 - protein, 18 aa, Homo sapiens derivative
CLGLSYDGLLHWDFAWPW

SEQ ID NO: 479 - protein, 26 aa, Homo sapiens derivative
HWDFAWPWCLGLSYDGLLHWDFAWPW

SEQ ID NO: 480 - protein, 21 aa, Homo sapiens derivative
  VARIANT (11)...(13): Any amino acid residue
CLGLSYDGLLXXXWPWAFDWH

SEQ ID NO: 481 - protein, 32 aa, Homo sapiens derivative
  VARIANT (9)...(11): Any amino acid residue
  VARIANT (22)...(24): Any amino acid residue
HWDFAWPWXXXCLGLSYDGLLXXXWPWAFDWH

SEQ ID NO: 482 - protein, 18 aa, Homo sapiens derivative
CLGLSYDGLLWPWAFDWH

SEQ ID NO: 483 - protein, 26 aa, Homo sapiens derivative
HWDFAWPWCLGLSYDGLLWPWAFDWH

SEQ ID NO: 484 - protein, 21 aa, Homo sapiens derivative
  VARIANT (9)...(11): Any amino acid residue
HWDFAWPWXXXLLKYRAREPV

SEQ ID NO: 485 - protein, 21 aa, Homo sapiens derivative
  VARIANT (11)...(13): Any amino acid residue
LLKYRAREPVXXXHWDFAWPW

SEQ ID NO: 486 - protein, 32 aa, Homo sapiens derivative
  VARIANT (9)...(11): Any amino acid residue
  VARIANT (22)...(24): Any amino acid residue
HWDFAWPWXXXLLKYRAREPVXXXHWDFAWPW

SEQ ID NO: 487 - protein, 18 aa, Homo sapiens derivative
HWDFAWPWLLKYRAREPV

SEQ ID NO: 488 - protein, 18 aa, Homo sapiens derivative
LLKYRAREPVHWDFAWPW

SEQ ID NO: 489 - protein, 26 aa, Homo sapiens derivative
HWDFAWPWLLKYRAREPVHWDFAWPW

SEQ ID NO: 490 - protein, 21 aa, Homo sapiens derivative
  VARIANT (11)...(13): Any amino acid residue
LLKYRAREPVXXXWPWAFDWH

SEQ ID NO: 491 - protein, 32 aa, Homo sapiens derivative
  VARIANT (9)...(11): Any amino acid residue
  VARIANT (22)...(24): Any amino acid residue
HWDFAWPWXXXLLKYRAREPVXXXWPWAFDWH

SEQ ID NO: 492 - protein, 18 aa, Homo sapiens derivative
LLKYRAREPVWPWAFDWH

SEQ ID NO: 493 - protein, 26 aa, Homo sapiens derivative
HWDFAWPWLLKYRAREPVWPWAFDWH

SEQ ID NO: 494 - protein, 20 aa, Homo sapiens derivative
  VARIANT (9)...(11): Any amino acid residue
HWDFAWPWXXXFLWGPRALV

SEQ ID NO: 495 - protein, 20 aa, Homo sapiens derivative
  VARIANT (10)...(12): Any amino acid residue
FLWGPRALVXXXHWDFAWPW

SEQ ID NO: 496 - protein, 31 aa, Homo sapiens derivative
  VARIANT (9)...(11): Any amino acid residue
  VARIANT (21)...(23): Any amino acid residue
HWDFAWPWXXXFLWGPRALVXXXHWDFAWPW

SEQ ID NO: 497 - protein, 17 aa, Homo sapiens derivative
HWDFAWPWFLWGPRALV

SEQ ID NO: 498 - protein, 17 aa, Homo sapiens derivative
FLWGPRALVHWDFAWPW

SEQ ID NO: 499 - protein, 25 aa, Homo sapiens derivative
HWDFAWPWFLWGPRALVHWDFAWPW

SEQ ID NO: 500 - protein, 20 aa, Homo sapiens derivative
  VARIANT (10)...(12): Any amino acid residue
FLWGPRALVXXXWPWAFDWH

SEQ ID NO: 501 - protein, 31 aa, Homo sapiens derivative
  VARIANT (9)...(11): Any amino acid residue
  VARIANT (21)...(23): Any amino acid residue
HWDFAWPWXXXFLWGPRALVXXXWPWAFDWH

SEQ ID NO: 502 - protein, 17 aa, Homo sapiens derivative
FLWGPRALVWPWAFDWH

(19) United States
(12) Patent Application Publication
(10) Pub. No.: US 2004/0101532 A1
(43) Pub. Date: May 27, 2004

(54) Methods and compositions for heat shock protein mediated immunotherapy of melanoma

(76) Inventors: Alan Houghton, New York, NY (US); Philip Livingston, New York, NY (US); Qais Al-Awqati, New York, NY (US); Mark Mayhew, New York, NY (US); Mee Hoe, Irvington, NY (US)

Correspondence Address: KENYON & KENYON, ONE BROADWAY, NEW YORK, NY, 10004, US

(21) Appl. No.: 10/258,144

(22) PCT Filed: Apr. 17, 2001
(86) PCT No.: PCT/US01/12449

(51) Int. Cl. A61K 39/00; A61K 38/08

(52) U.S. Cl. 424/185.1; 514/15

(57) ABSTRACT

The present invention relates to immunotherapeutic compositions comprising an effective amount of a molecular chaperone such as a heat shock protein, preferably hsp70, non-covalently bound to one or more javelinized melanoma antigens and to methods of using the immunotherapeutic compositions to induce an immune response against melanoma in a subject. The immunotherapeutic composition may contain one or more heat shock proteins, such as one or more of hsp70, hsp90, gp96, BiP, and hsp40, and may contain one or more javelinized melanoma antigens.

### 1. INTRODUCTION

The present invention relates to novel compositions and methods for treating or preventing malignant melanoma, whereby a subject is immunized with a composition comprising one or more melanoma antigen bound, via a molecular tether referred to as a “javelin”, to a heat shock protein.

### 2. BACKGROUND OF THE INVENTION

### 2.1 Melanoma

Malignant melanoma is the most serious form of skin cancer, accounting for two percent of all cancers. It is currently the eighth most common cancer in the United States, with about 47,000 new cases per year of melanoma in the United States alone.

The American Joint Committee on Cancer (“AJCC”) has developed a staging system (currently under revision) which classifies melanoma according to thickness (depth) of the tumor and the extent to which it has spread. The AJCC has proposed a new set of criteria which also consider, among other things, whether or not a melanoma is ulcerated and the number, rather than the gross dimensions, of metastatic lymph nodes (Balch et al., 2000, Cancer 88(6):1484-1491).

Surgery can be curative for the earliest stages of melanoma, and wide excision of a primary cutaneous melanoma is associated with a 10-year cure rate of 85 percent when the tumor's depth is less than 1.5 mm (AJCC Stage I). However, according to Barth and Morton, 1995, Cancer 15 (2 Suppl):726-734, recurrence will occur in 50 percent of patients with deep (>4 mm) primary melanomas, 60-85 percent of patients with regional lymph node metastases (AJCC Stage III) and 95 percent of patients with distant metastases (AJCC Stage IV). Even melanoma patients with AJCC Stage III or Stage IV disease who are free of detectable disease or have minimal disease have a less than 50% chance of surviving five years with currently available therapy. (Parker et al., 1997, Cancer Statistics 47:5-27, 1997; Houghton, 1992, Cutaneous Melanoma, 2 ed., J. B. Lippincott, pp. 499-508; Sirott et al., 1993, Cancer 72:3091-3098). Therefore, research efforts have been directed toward developing adjuvant therapies for melanoma.

According to Agarwala and Kirkwood, 2000, Forum (Genova) 10(3):230-239, although a number of agents have been tested for the adjuvant therapy of high risk melanoma, the only one to demonstrate an improvement in relapse-free and overall survival is interferon-alpha 2b, administered at maximally tolerated doses. Interferon gamma has been observed to exert certain effects which are similar to those produced by interferon alpha on melanoma cells in culture (Heninger et al., 2000, Inflamm. Res. 49(8):393-397), and clinical trials are under way in which a retroviral vector carrying an interferon gamma gene is being delivered into the tumors of patients with metastatic melanoma (Fujii et al., 2000, Cancer Gene Ther. 7(9):1220-1230).

### 2.2 Melanoma Antigens

Melanoma antigens, including gangliosides such as GM2 and GD3 and various peptides, have been identified that can act as specific targets for immune recognition and destruction of melanoma cells (Cebon et al., 1997, Austral. J. Dermatol. 38(Suppl 1):S66-S72).

The development of effective vaccines or immunotherapies for melanoma is dependent on the generation of protective immune responses to melanoma antigens.

Peptide antigens on the melanoma cell surface in association with Human Leukocyte Antigen (“HLA”) molecules have been identified which are recognized by cytotoxic lymphocytes (Cebon et al., 1997, Austral. J. Dermatol. 38(Suppl 1):S66-S72). Examples of specific antigens include MAGE proteins, tyrosinase, tyrosinase related proteins 1 and 2 (“TRP-1” and “TRP-2”), MelanA/MART-1, gp100, NY-ES01, BAGE-, GAGE-1/2 and others (see Dalerba et al., 1998, Int. J. Cancer 77(2 :200-204; Boel et al., 1995, Immunity 2:167-175). There have been a number of reports relating to the evaluation of melanoma associated peptide-based vaccines as a treatment modality (Rosenberg et al., 1998, Nature Medicine 4:321-327; Lewis et al., 2000, Int. J. Cancer 87:391-398; Jaeger et al., 1996, Int. J. Cancer 66:162-169; Nestle et al., 1998, Nature Medicine 4:328-332).

MAGE (Melanoma Antigen GEnes) proteins are encoded by a family of at least twenty-one related genes, including MAGE-1 to −12 (now named MAGE-A1-A12, MAGE-B1 to B4, and MAGE-C1), and four newly identified MAGE genes, namely MAGE-B5, MAGE-B6, MAGE-C2 and MAGE-C3 (Lucas et al., 2000, Int. J. Cancer 87(1):55-60). Genes of this family are expressed in various tumors of different histological types but are silent in normal tissues, with the exception of male germ line cells (which lack HLA expression) and placenta. Although many of these genes are selectively expressed in melanoma cells, Gibbs et al., 2000, Melanoma Res. 10(3):259-264 reports that MAGE-12 and MAGE-6 were expressed at higher frequencies (74 percent and 64 percent, respectively) than the other MAGE genes.

Tyrosinase is the rate-limiting enzyme in melanin synthesis and is a melanoma associated antigen that is recognized, in an HLA-restricted manner, by CD4+ and CD8+ T lymphocytes (Fetsch et al., 2000, Cancer 90(4:252-257). Tyrosinase-derived peptides are currently being utilized as targets for T cells in several immunotherapy protocols for metastatic malignant melanoma at the National Institutes of Health/National Cancer Institute of the United States (Id.).

In addition to tyrosinase, two tyrosinase family antigens associated with melanoma have been identified, known as Tyrosine Related Proteins 1 and 2 (“TRP-1 and TRP-2”). Immunization of mice with TRP-1 (Vuayasaradhi and Houghton, 1991, Int. J. Cancer 47:298-303; TRP-1 is also referred to in the literature as gp75 or the brown locus protein) has been observed to induce tumor immunity and autoimmunity (manifested as depigmentation) that is mediated by autoantibodies (Bowne et al., 1999, J. Exp. Med. 190(11):1717-1722, citing Weber et al., 1998, J. Clin. Invest. 102:1258-1264; Hara et al., 1995, J. Exp. Med. 182:1609-1614; Nafzger et al., 1996, Proc. Natl. Acad. Sci. U.S.A. 93:14809-14814; and Clynes et al., 1998, Proc. Natl. Acad. Sci. U.S.A. 95:652-656).

TRP-2 (also known as the slaty locus protein) has been found to frequently be expressed in melanoma cells (Noppen et al., 2000, Int. J. Cancer 87(2):241-246). Seven HLA-A*0201-restricted TRP-2 peptides were found to bind to HLA-A*0201 binding motifs by computer-assisted reverse immunology; of these seven, two, TRP-2(360-368) and TRP-2(476-484) induced specific CD8+ cytotoxic T lymphocytes (Id.). An additional epitope-containing peptide, TRP-2(288-296), was identified by Sun et al., 2000, Int. J. Cancer 87(3):399-404.

MART-1 (Melanoma Antigen Recognized by T-Cells-1) is a melanocyte differentiation antigen (U.S. Pat. No. 5,874,560 by Kawakami, Brinckerhoffet al., 1999, Int. J. Cancer 83(3):326-334; Zarour et al., 2000, Proc. Natl. Acad. Sci. U.S.A. 97(1):400-405; Kawakami et al., 1994, Proc. Natl. Acad. Sci. U.S.A. 91:6458-6462; Castelli et al., 1995, J. Exp. Med. 181(1):363-368). The human Melan-A/MART-1 gene encodes an HLA-A2-restricted peptide epitope recognized by melanoma-reactive CD8+ cytotoxic T lymphocytes, but Zarour et al., 2000, Proc. Natl. Acad. Sci. U.S.A. 97(1):400-405 have identified a peptide fragment of MART-1 which is HLA-DR4 presented and recognized by CD4+ T lymphocytes.

The gp100 gene was localized to chromosome 12p-q21 and encodes a glycoprotein, recognized by monoclonal antibody HMB-45, that is mainly localized in the membrane and filamentous matrix of pre-melanosomes, suggesting that it may be involved in melanin synthesis. Gp100 is recognized as a melanoma-associated antigen, and gp 100 mRNA has been reported to be a more sensitive marker than tyrosinase mRNA for detected circulating melanoma cells in peripheral blood (Tsukamoto et al., 2000, J. Dermatol. 23(2):126-131).

The ability of melanoma antigen-reactive T cells to mediate in vivo tumor regression has been observed in murine tumor models and by occasional clinical responses to adoptive immunotherapy using tumor infiltrating lymphocytes isolated from patients with melanoma. (Kawakami et al., 1994, Proc. Natl. Acad. Sci USA 91:6458-6462; Kawakami et al., 1995, J. Immunol. 154: 3961-3968).

In order to induce a T cell response, certain antigens must be presented by major histocompatability complex (MHC) molecules. Molecular chaperones and heat shock proteins (“hsps”) are able to deliver bound antigens to the antigen presenting cells (“APCs”) for subsequent display on MHC Class I or MHC Class II molecules, which in turn, may lead to the generation of a T cell response. (Schild et al., 1999, Current Opinion in Immunology 11:109-113). Bullock et al., 2000, J. Immunol. 164(5):2354-2361 report that the density of tyrosinase and gp100 peptides displayed by dendritic cells, a type of APC, affects the size of the CD8+ cytotoxic T cell population activated.

However, many antigens do not bind sufficiently well to heat shock proteins or other molecular chaperones for them to be efficiently delivered to APCs. It has been discovered that attaching certain molecules, particularly specific peptides, to an antigen can increase the affinity of the antigen toward an hsp or other molecular chaperone. This process, termed “javelinization”, is described in International Patent Application Nos. PCT/US96/13363 and PCT/US98/22335 by Rothman et al., inventors. According to the present invention, this process of “javelinization” is used to promote the association between melanoma antigens and heat shock proteins in novel methods for the treatment of melanoma.

### 3. SUMMARY OF THE INVENTION

The present invention relates to immunotherapeutic compositions comprising an effective amount of a heat shock protein, preferably hsp70, non-covalently bound to one or more javelinized melanoma antigens and to methods of using the immunotherapeutic compositions to induce an immune response against melanoma in a subject. The immunotherapeutic composition may contain one or more heat shock proteins, such as one or more of hsp70, hsp90, gp96, BiP, hspl 70 and hsp40, and may contain one or more javelinized melanoma antigens.

### 4. DETAILED DESCRIPTION OF THE INVENTION

The present invention provides a method of producing an immune response in a subject against melanoma antigens. This is achieved by the general step of immunizing the subject with an immunotherapeutic composition comprising one or more heat shock proteins non-covalently bound to one or more javelinized melanoma antigens. The heat shock proteins are preferably mammalian heat shock proteins, and preferably originate from the same species as that of the subject to be treated.

### 4.1.1 Melanoma Antigens

A “melanoma antigen”, as that term is used herein, is any molecule (including a protein, lipid, or carbohydrate or a combination or derivative thereof) which can induce a selective immune response against melanoma cells. An immune response is selective against melanoma cells if it is directly cytotoxic (e.g., via cytotoxic T cells or natural killer cells) to melanoma cells or indirectly cytotoxic (e.g., antibody directed cellular cytotoxicity) to melanoma cells but not substantially directly or indirectly cytotoxic to non-melanoma cells.

The immune response is considered to be substantially directly or indirectly cytotoxic to melanoma cells if the response may be clinically correlated with a slowing or arrest of progression of melanoma tumor growth or spread.

The immune response is considered to be directly or indirectly cytotoxic to non-melanoma cells in a subject if unacceptable clinical toxicity is observed. The toxic side effects, and the extent of toxic effects, of cancer chemotherapeutic agents approved by the United States Food and Drug Administration used in their standard doses may be considered to represent acceptable clinical toxicity. Acceptable clinical toxicity may occur if, for example, a melanoma antigen is expressed at low levels in a non-melanoma cell (defined as any somatic or germ cell other than a melanoma cell, including, but not limited to, melanocytes and other pigment-containing cells) but at higher levels in a melanoma cell.

Melanoma antigens may be derived from malignant tissue or cell lines or be prepared by chemical and/or recombinant methods. The melanoma antigen may comprise one or more melanoma selective epitopes and may also comprise non-immunogenic structures. The epitope may, in some non-limiting embodiments, be characterized (e.g., its chemical structure and/or peptide sequence may be known). Alternatively, the epitope(s) may be uncharacterized, but presumed to be present in the molecule because that molecule induces a melanoma selective immune response in an appropriate subject.

For example, the melanoma antigen of the invention may comprise elements such that it is MHC-restricted, in that its capacity to induce an immune response is restricted to a particular MHC class I or II type (see Kubo et al., 1994, J. Immun. 152:3913). Under such circumstances, the melanoma antigen may be engineered to comprise one or more natural or heteroclitic MHC class I and/or MHC class II binding peptides, where the binding peptide may be distinct from the epitope(s) or the same peptide may serve as both epitope and binding peptide. Such an antigen is desirably administered to subjects manifesting the appropriate MHC type. The need for incorporating an MHC binding peptide may vary depending on the nature and number of epitopes comprised in the melanoma protein antigen; Yang et al., 2000, J. Immunol. 164(8):4202-4211 report that genetically modified dendritic cells that express an entire melanoma protein antigen present a wide array of possible CTL epitopes and may therefore be substantially less HLA restricted than smaller peptide antigens. It may be desirable to select or modify antigens to bind to HLA supertypes, where a “supertype” is a group of HLA types which exhibit overlapping peptide-binding repertoires (Sette and Sidney, 1998, Curr. Opin. Immunol. 10:478-482). The MHC restriction of several of the peptide antigens provided for herein is specified below.

In alternative embodiments, the melanoma antigens are not restricted to one HLA type but are recognizable by a plurality of HLA-types for induction of an immune response.

In one set of non-limiting embodiments of the invention, the melanoma antigen may be a ganglioside, including, but not limited to, GM2 (see Livingston, 1998, Semin. Oncol. 25(6):636-645) or GD3.

In another set of non-limiting embodiments, the melanoma antigen may be a protein or a peptide. A “protein antigen” is a molecule comprising more than 50 amino acid residues. A “peptide antigen” is a molecule comprising between 5 and 49 amino acid residues. The desirable size of a melanoma antigen may vary depending upon the number and chemical nature of the above-mentioned elements comprised. For example, melanoma selective CTL epitopes according to the invention typically comprise between 8 and 15, and preferably between 8 and 10, amino acids, so that a melanoma antigen according to the invention which comprises a CTL epitope desirably includes at least 8 amino acids, and preferably between 8 and 15 amino acids. Where the melanoma antigen is a protein, it may be a naturally occurring protein or a variant thereof or may be a fusion protein.

Protein antigens and peptide antigens, as those terms are used herein, encompass molecules which have been modified or naturally contain carbohydrate or lipid residues, non-naturally occurring amino acids, amino acid analogs, or other covalently linked molecules, such as a benzoquinone ansamycin antibiotic or a portion thereof.

Specific non-limiting examples of such modifications include those which improve stability of the protein or peptide. For example, but not by way of limitation, Brinckerhoffet al., 1999, Int. J. Cancer 83(3:326-334 report that terminal modifications inhibit proteolytic degradation of an immunogenic MART-1 (27-35) peptide. Miconnet et al., 2000, J. Biol. Chem. 275(35):26892-26897 report that amino acid identity or position determines the proteosomal cleavage of the MAGE-3 peptide (271-279). Other specific non-limiting examples of such modifications include those which enhance the ability of a melanoma antigen to produce an immune response. For example, Minev et al., 2000, Eur. J. Immunol. 30(8):2115-2124 report that the addition of synthetic signal sequences at the N-terminus of MART-1 enhanced MHC class I presentation. Ayyoub et al., 1999, J. Biol. Chem. 274:10227-10234 describes “a structure-based approach to designing non-natural peptides that can activate anti-melanoma cytotoxic T cells”. Guichard et al., 2000, J. Med. Chem. 43(20):3803-3808 report results that suggest that substitution of a plurality of amino acids and the use of beta-amino acid substituents may be useful modifications for increasing MHC binding capacity. It should be noted, however, as stated in De Berardinis et al., 1997, Human Immunol. 54:189-193, that altering the sequences bordering epitopes may alter the composition of the population of T cells primed, relative to the naturally occurring epitope.

As regards specific peptides set forth in the following sections, variants may be created by amino acid modifications or by genetically engineering substitutions, deletions, insertions or additions to the naturally occurring sequence. The naturally occurring peptide or protein may, for example, be modified by substituting one or more amino acids with a non-naturally occurring amino acid or an amino acid analog. Further, the naturally occurring or variant peptide or protein may be conjugated to a second molecule, such as a peptide or protein, a carbohydrate, a lipid, or a benzoquinone ansamycin antibiotic.

The present invention further provides for fragments of the specifically recited antigens provided that they retain the melanoma specific or selective epitope; the skilled artisan would be able to identify the epitope-containing region using standard techniques. In addition, as set forth below, a melanoma antigen is modified in the sense that it is conjugated to a javelin molecule.

A melanoma antigen for use according to the invention may be identified by any method known in the art (see, for example, Reynolds et al., 2000, J. Immunol. Methods 244:59-67 and Noppen et al., 2000, Int. J. Cancer 87(2:241-246). Non-limiting examples of suitable melanoma antigens described in the following subsections include the melanoma protein antigens tyrosinase, tyrosinase-related proteins 1 and 2, gp100, MART, MAGE and BAGE antigens, and NY-ES01, as well as epitope-containing peptide fragments thereof. Alternatively, the melanoma antigen may be a variant of a tyrosinase, tyrosinase related protein 1 or 2, gp100, MART, BAGE, GAGE, NY-ES01 or MAGE protein or an epitope-containing fragment thereof.

### 4.1.2. Tyrosinase

Tyrosinase (EC 1.14.18.1) is a melanosomal glycoprotein that is essential in melanin synthesis. The tyrosinase gene, originally cloned at Memorial Sloan Kettering Cancer Center (MSKCC) consists of five exons and is localized to chromosome 11q14-q21. The GeneBank Accession Number for the human tyrosinase sequence is XM 006020. See Bouchar et al., 1989, J. Exp. Med. 169:2029; see also Kwon et al., 1987, Proc. Natl. Acad. Sci. U.S.A. 84:7473-7477 and Ponnazhagan et al., 1994, J. Invest. Dermatol. 102:744-748. Immunophenotyping of melanomas for tyrosinase is described in Chen et al., 1995, Proc. Natl. Acad. Sci. U.S.A. 92:8125-8129. Any epitope of tyrosinase which falls within the definition of a melanoma specific antigen set forth herein may be utilized according to the invention.

Tyrosinase has been reported to be recognized by cytotoxic T cells from melanoma patients, and peptides from the signal peptide and from the catalytic domain have been implicated as being particularly relevant (Brichard et al., 1993, J. Exp. Med. 178:489-495; Robbins et al., 1994, Cancer Res. 54:3124-3126; Wolfel et al., 1994, Eur. J. Immunol. 24:759-764).

In preferred embodiments of the invention, one or both of the following previously described epitope-containing peptides (presented as the single letter code for amino acids) may be utilized as melanoma antigens: YMNGTMSQV (SEQ ID NO: 1; Wolfel et al., 1994, Eur. J. Immunol. 24:759-764) and/or MLLAVLYVL (SEQ ID NO: 2; Skipper et al.,1996, J. Exp. Med. 183(2):527-534), or epitope-containing fragments thereof. It should be noted that peptides may be referred to herein in the alternative by either the single letter or three letter code.

In another specific embodiment of the invention, a modification of YMNGTMSQV (SEQ ID NO: 1) may be used, namely YMDGTMSQV (SEQ ID NO: 3) (or an epitope-containing fragment thereof), termed tyrosinase:368-376 (370D), which corresponds to amino acids 368 to 376 of the tyrosinase protein modified at 370th amino acid position to substitute aspartic acid for asparagine. This peptide has been tested in a clinical trial and was well tolerated with little toxicity. Two of nine vaccinated patients showed an increase in T cell responsiveness against tryosinase after immunization with the tyorinase peptide antigen by the ELISPOT assay. (Lewis et al.,2000, Int. J. Cancer 87(3):391-398).

### 4.1.3. Tyrosinase Related Proteins

Tyrosine related proteins, such as the differentiation antigens TRP-1 (GeneBank Accession No XM 005426) and TRP-2 (GeneBank Accession No D17547; Yokoyama et al., 1994), S69231 (Bouchard et al., 1994), and epitope-containing peptide portions thereof, may also be used as melanoma antigens. Specific nonlimiting examples of TRP-2 peptides containing melanoma specific/selective epitopes which may be used according to the invention include: ORF3P (TRP-1) MSLQRQFLR (SEQ ID NO: 4; Coulie et al., 1995, Proc. Natl. Acad. Sci. U.S.A. 92:7976-7980); TRP-2(180-188) SVYDFFVWL (SEQ ID NO: 5; Bowne et al., 1999, J. Exp. Med. 190:1717-1722); TRP-2 (197-205) LLGPGRPYR (SEQ ID NO: 6; Wang et al., 1996, J. Exp. Med. 184:2207-2216); TRP-2(288-296), SLDDYNHLV (SEQ ID NO: 7; Sun et al., 2000, Int. J. Cancer 87(3):399-404; HLA-A*0201-restricted), TRP-2(360-368) TLDSQVMSL (SEQ ID NO: 8; Noppen et al., 2000, Int. J. Cancer 87(2):241-246; HLA-A*0201-restricted) and TRP-2(476-484) VMGTLVALV (SEQ ID NO: 9; Noppen et al., 2000, Int. J. Cancer 87(2):241-246; HLA-A*0201-restricted), or modified versions or epitope-containing fragments thereof

### 4.1.4.GP100

Any epitope of gp100 (GeneBank Accession No. S73003) which falls within the definition of a melanoma specific antigen set forth herein may be utilized according to the invention. In nonlimiting embodiments of the invention, gp100 is primarily recognized in the context of HLA-A*0201.

Nonlimiting examples of suitable peptide antigens derived from the gp100 melanoma antigen include ITDQVPFSV (SEQ ID NO: 10), TITDQVPFSV (SEQ ID NO: 11), YLEPGVTVA (SEQ ID NO: 12), YLEPGVTVA (SEQ ID NO: 13), KTWGQYWQV (SEQ ID NO:14), TWGQYWQVL (SEQ ID NO:15), VLKRCLLHL (SEQ ID NO: 16), LNVSLADTN (SEQ ID NO: 17), SLADTNSLAV (SEQ ID NO: 18), LLDGTATLRL (SEQ ID NO: 19), VLYRYGSFSV (SEQ ID NO: 20), ALDGGNKHFL (SEQ ID NO: 21), and VLPSPACQLV (SEQ ID NO: 22) and epitope-containing fragments thereof (see U.S. Pat. No. 5,844,075). Regarding peptide antigens, see also Yang et al., 2000, J. Immunol. 164(8):4204-4211.

In a preferred nonlimiting embodiment of the invention, one of the epitope-containing peptides, ITDQVPFSV (SEQ ID NO: 10) has been modified at the second position, corresponding to gp100:209-217, to substitute methionine for threonine and yield IMDQVPFSV (SEQ ID NO: 23) to increase the affinity of binding to HLA-A0201. (Parkhurst et al., 1996, J. Immunol. 157(6):2539-2548). Immunization with this peptide has been reported to induce anti-gp 100 T cell reactivity that could be detected without extensive preliminary in vitro sensitization suggesting that immunization with this peptide can efficiently expand the population of anti-gp100 T cells.

### 4.1.5. MART-1/Melan A

MART-1 (Melanoma Antigen Recognized by T-Cells-1)(GeneBank Accession No. U06452 for MART, and, for Melan A, NM 005511 and XM005519) is a melanocyte differentiation antigen (U.S. Pat. No. 5,874,560 by Kawakami, Brinckerhoff et al., 1999, Int. J. Cancer 83(3):326-334; Zarour et al., 2000, Proc. Natl. Acad. Sci. U.S.A. 97(1):400-405; Kawakami et al., 1994, Proc. Natl. Acad. Sci. U.S.A. 91:6458-6462; Castelli et al.,1995, J. Exp. Med. 181(1):363-368). Any epitope of MART-1 which falls within the definition of a melanoma specific antigen set forth herein may be utilized according to the invention.

Particular peptides which include melanoma specific epitopes include MART-1 (51-73) and MART-1 (27-35) (AAGIGILTV; SEQ ID NO:24), which may be sources of melanoma antigens. Nonlimiting examples of MART-1 peptide antigens derived from the MART-1 protein antigen include ILTVILGVL (SEQ ID NO: 25), EAAGIGILTV (SEQ ID NO: 26) and AAGIGILTVI (SEQ ID NO: 27) (Kawakami et al., 1994, J. Exp. Med. 180:347-352), or epitope-containing fragments thereof A further example is [Leu(28), beta-HIle(30)]MART-1 (27-35), as described in Guichard et al., 2000, J. Med. Chem. 43(20):3803-3808.

### 4.1.6. Mage Antigens

MAGE antigens include the products of a gene family including MAGE-1 to −12 (now named MAGE-A1-A12, MAGE-B1 to B4, and MAGE-C1), and four newly identified MAGE genes, namely MAGE-B5, MAGE-B6, MAGE-C2 and MAGE-C3 (Lucas et al., 2000, Int. J. Cancer 87(1):55-60), as well as genes homologous thereto which are considered to be included in the MAGE gene family. Any epitope of a protein of the MAGE family which falls within the definition of melanoma specific antigen set forth herein may be utilized according to the invention. Gibbs et al., 2000, Melanoma Res. 10(3:259-264 report that MAGE-12 and MAGE-6 are expressed by melanoma cells at higher frequencies than other MAGE genes, and therefore may be particularly useful. The GeneBank Accession Numbers are, for MAGE A1: NM 004988; MAGE A2: NM 005361; MAGE A3: NM 020017; MAGE A4: NM 002362; MAGE A6: NM 005363; MAGE A12: NM 005367.

Non-limiting examples of peptide antigens derived from MAGE-1 protein antigen include ALEAQQEAL (SEQ ID NO: 28), ILESLFRAV (SEQ ID NO: 29), SLHCKPEEAL (SEQ ID NO: 30), PLVLGTLEEV (SEQ ID NO: 31) and epitope-containing fragments thereof. Non-limiting examples of peptide antigens derived from MAGE-1/3 include CLGLSYDGL (SEQ ID NO: 32), LLKYRAREPV (SEQ ID NO: 33) and epitope-containing fragments thereof; and nonlimiting examples of peptide antigens derived from MAGE-3 include MAGE-3(271-279), particularly FLWGPRALV (SEQ ID NO:34) and epitope-containing fragments thereof. (Miconnet et al., 2000, J. Biol. Chem. 275:26892-26897; International Patent Application No. PCT/US94/02353 by Cytel, Van der Bruggen et al. inventors). Another example of a peptide antigen that may be used according to the invention is the MAGE-A1 peptide EADPTGHSY (SEQ ID NO: 35), which is HLA-A1 and HLA-B35 restricted (Luiten et al., 2000, Tissue Antigens 56(1):77-81).

### 4.1.7. Javelinization of Antigens

Heat shock protein 70 (“hsp70”) has been shown to be effective at delivering bound peptide antigens to antigen presenting cells for their display on MHC class I molecules. Immunization of mice with hsp70-bound antigens has resulted in the generation of strong cellular immune responses against the chosen antigen. However, in vitro experimentation has shown that many optimal MHC class I binding antigens do not bind well to hsp70. Binding of diverse antigens to various heat shock proteins can be facilitated by “javelinization” (Moroi et al., 2000, Proc Natl. Acad. Sci. U.S.A. 97(7):3485-3490).

The term “javelin” as used herein refers to a molecule which itself is capable of non-covalently binding to a heat shock protein, and which, when covalently linked to a melanoma antigen, acts as a tether, creating a non-covalent physical association between the melanoma antigen and the heat shock protein.

The javelin may be a member of any class of biochemical molecule or combination thereof, but is preferably a peptide or a peptidomimetic compound. The particular structure of a javelin will depend, to at least some degree, on the heat shock protein to which it binds. It should be noted, however, that because particular heat shock proteins act as molecular chaperones in the process of protein folding, they are typically capable of binding to a variety of javelin molecules. Suitable javelin molecules, and methods for identifying further javelin molecules, are described in co-pending International Patent Application No. PCT/US98/22335, incorporated by reference in its entirety herein.

Accordingly, the javelin to be covalently linked to a melanoma antigen is chosen based on the particular heat shock protein or heat shock proteins to which it is intended to bind. Such heat shock protein may be any known or yet to be identified heat shock protein or portion thereof, or any fusion protein comprising at least a portion of a heat shock protein. The term “heat shock protein”, as used herein, refers to stress proteins (including homologs thereof expressed constitutively), including, but not limited to, gp96, hsp170, hsp90, BiP, hsp70, hsp60, hsp40, hsc70, and hsp10. Hsp target may be prepared from a natural source, expressed recombinantly, or chemically synthesized.

In particular, non-limiting embodiments of the invention, javelins may have amino acid compositions which comprise a substantial proportion of hydrophobic amino acids such as phenylalanine and tryptophan, and to a lesser extent, leucine and/or a substantial number of serine, threonine, or proline residues. In particular, nonlimiting embodiments, javelins of the invention may comprise amino acid sequences which have the general description hydrophobic—basic—hydrophobic—hydrophobic—hydrophobic; Ser/Thr—hydrophobic—hydrophobic—Ser/Thr; Ser/Thr—Ser/Thr—hydrophobic—hydrophobic—Ser/Thr—Ser/Thr; and Ser/Thr—Ser/Thr—Hydrophobic—hydrophobic—hydrophobic. Alternatively, javelins may comprise heat shock binding peptides as described in Blond-Elguindi et al., 1993, Cell 75:717-728, including the consensus sequence hydrophobic—(Trp/Xaa)—hydrophobic—Xaa—hydrophobic—Xaa—hydrophobic (where Xaa may be any amino acid) and the specific peptides His Trp Asp Phe Ala Trp Pro Trp (SEQ ID NO: 36) and Phe Trp Gly Leu Trp Pro Trp Glu (SEQ ID NO: 37); Auger et al., 1996, Nature Med. 2:306-310, including Gin Lys Arg Ala Ala (SEQ ID NO:38) and Arg Arg Arg Ala Ala (SEQ ID NO:39); Flynn et al., 1989, Science 245:385-390; Gragerov et al., 1994, J. Mol. Biol. 235:848-854; Terlecky et al., 1992, J. Biol. Chem. 267:9202-9202, Lys Phe Glu Arg Gin (SEQ ID NO: 40); and Nieland et al., 1996, Proc. Natl. Acad. Sci. U.S.A. 93:6135-6139, including the VSV8 peptide, Arg Gly Tyr Val Tyr Gin Gly Leu (SEQ ID NO: 41). In preferred embodiments, javelins of the invention may have a length of 4-50 amino acid residues, and more preferably 7-20 amino acid residues.

In specific, non-limiting embodiments, the following amino acid sequences, discussed more fully in International Patent Application No. PCT/US98/22335, may be covalently linked to melanoma antigens according to the invention:

| | |
|---|---|
| Tyr Thr Leu Val Gln Pro Leu; | (SEQ ID NO: 42) |
| Thr Pro Asp Ile Thr Pro Lys; | (SEQ ID NO: 43) |
| Thr Tyr Pro Asp Leu Arg Tyr; | (SEQ ID NO: 44) |
| Asp Arg Tbr His Ala Thr Ser; | (SEQ ID NO: 45) |
| Met Ser Tbr Thr Phe Tyr Ser; | (SEQ ID NO: 46) |
| Tyr Gln His Ala Val Gln Thr; | (SEQ ID NO: 47) |
| Phe Pro Phe Ser Ala Ser Thr; | (SEQ ID NO: 48) |
| Ser Ser Phe Pro Pro Leu Asp; | (SEQ ID NO: 49) |
| Met Ala Pro Ser Pro Pro His; | (SEQ ID NO: 50) |
| Ser Ser Phe Pro Asp Leu Leu; | (SEQ ID NO: 51) |
| His Ser Tyr Asn Arg Leu Pro; | (SEQ ID NO: 52) |
| His Leu Tbr His Ser Gln Arg; | (SEQ ID NO: 53) |
| Gln Ala Ala Gln Ser Arg Ser; | (SEQ ID NO: 54) |
| Phe Ala Thr His His Ile Gly; | (SEQ ID NO: 55) |
| Ser Met Pro Glu Pro Leu Ile; | (SEQ ID NO: 56) |
| Ile Pro Arg Tyr His Leu Ile; | (SEQ ID NO: 57) |
| Ser Ala Pro His Met Thr Ser; | (SEQ ID NO: 58) |
| Lys Ala Pro Val Trp Ala Ser; | (SEQ ID NO: 59) |
| Leu Pro His Trp Leu Leu Ile; | (SEQ ID NO: 60) |
| Ala Ser Ala Gly Tyr Gln Ile; | (SEQ ID NO: 61) |
| Val Thr Pro Lys Tbr Gly Ser; | (SEQ ID NO: 62) |
| Glu His Pro Met Pro Val Leu; | (SEQ ID NO: 63) |
| Val Ser Ser Phe Val Thr Ser; | (SEQ ID NO: 64) |
| Ser Thr His Phe Thr Trp Pro; | (SEQ ID NO: 65) |
| Gly Gln Trp Trp Ser Pro Asp; | (SEQ ID NO: 66) |
| Gly Pro Pro His Gln Asp Ser; | (SEQ ID NO: 67) |
| Asn Thr Leu Pro Ser Thr Ile; | (SEQ ID NO: 68) |
| His Gln Pro Ser Arg Trp Val; | (SEQ ID NO: 69) |
| Tyr Gly Asn Pro Leu Gln Pro; | (SEQ ID NO: 70) |
| Phe His Trp Trp Trp Gln Pro; | (SEQ ID NO: 71) |
| Ile Thr Leu Lys Tyr Pro Leu; | (SEQ ID NO: 72) |
| Phe His Trp Pro Trp Leu Phe; | (SEQ ID NO: 73) |
| Thr Ala Gln Asp Ser Thr Gly; | (SEQ ID NO: 74) |
| Phe His Trp Trp Trp Gln Pro; | (SEQ ID NO: 75) |
| Phe His Trp Trp Asp Trp Trp; | (SEQ ID NO: 76) |
| Glu Pro Phe Phe Arg Met Gln; | (SEQ ID NO: 77) |
| Thr Trp Trp Leu Asn Tyr Arg; | (SEQ ID NO: 78) |
| Phe His Trp Trp Trp Gln Pro; | (SEQ ID NO: 79) |
| Gln Pro Ser His Leu Arg Trp; | (SEQ ID NO: 80) |
| Ser Pro Ala Ser Pro Val Tyr; | (SEQ ID NO: 81) |
| Phe His Trp Trp Trp Gln Pro; | (SEQ ID NO: 82) |
| His Pro Ser Asn Gln Ala Ser; | (SEQ ID NO: 83) |
| Asn Ser Ala Pro Arg Pro Val; | (SEQ ID NO: 84) |
| Gln Leu Trp Ser Ile Tyr Pro; | (SEQ ID NO: 85) |
| Ser Trp Pro Phe Phe Asp Leu; | (SEQ ID NO: 86) |
| Asp Thr Thr Leu Pro Leu His; | (SEQ ID NO: 87) |
| Trp His Trp Gln Met Leu Trp; | (SEQ ID NO: 88) |
| Asp Ser Phe Arg Thr Pro Val; | (SEQ ID NO: 89) |
| Thr Ser Pro Leu Ser Leu Leu; | (SEQ ID NO: 90) |
| Ala Tyr Asn Tyr Val Ser Asp; | (SEQ ID NO: 91) |
| Arg Pro Leu His Asp Pro Met; | (SEQ ID NO: 92) |
| Tip Pro Ser Thr Thr Leu Phe; | (SEQ ID NO: 93) |
| Ala Thr Leu Glu Pro Val Arg; | (SEQ ID NO: 94) |
| Ser Met Thr Val Leu Arg Pro; | (SEQ ID NO: 95) |
| Gln Ile Gly Ala Pro Ser Trp; | (SEQ ID NO: 96) |
| Ala Pro Asp Leu Tyr Val Pro; | (SEQ ID NO: 97) |
| Arg Met Pro Pro Leu Leu Pro; | (SEQ ID NO: 98) |
| Ala Lys Ala Thr Pro Glu His; | (SEQ ID NO: 99) |
| Thr Pro Pro Leu Arg Ile Asn; | (SEQ ID NO: 100) |
| Leu Pro Ile His Ala Pro His; | (SEQ ID NO: 101) |
| Asp Leu Asn Ala Tyr Thr His; | (SEQ ID NO: 102) |
| Val Thr Leu Pro Asn Phe His; | (SEQ ID NO: 103) |
| Asn Ser Arg Leu Pro Thr Leu; | (SEQ ID NO: 104) |
| Tyr Pro His Pro Ser Arg Ser; | (SEQ ID NO: 105) |
| Gly Thr Ala His Phe Met Tyr; | (SEQ ID NO: 106) |
| Tyr Ser Leu Leu Pro Thr Arg; | (SEQ ID NO: 107) |
| Leu Pro Arg Arg Thr Leu Leu; | (SEQ ID NO: 108) |
| Thr Ser Thr Leu Leu Trp Lys; | (SEQ ID NO: 109) |
| Thr Ser Asp Met Lys Pro His; | (SEQ lD NO: 110) |
| Thr Ser Ser Tyr Leu Ala Leu; | (SEQ ID NO: 111) |
| Asn Leu Tyr Gly Pro His Asp; | (SEQ ID NO: 112) |
| Leu Glu Thr Tyr Thr Ala Ser; | (SEQ ID NO: 113) |
| Ala Tyr Lys Ser Leu Thr Gln; | (SEQ ID NO: 114) |
| Ser Thr Ser Val Tyr Ser Ser; | (SEQ ID NO: 115) |
| Glu Gly Pro Leu Arg Ser Pro; | (SEQ ID NO: 116) |
| Thr Thr Tyr His Ala Leu Gly; | (SEQ ID NO: 117) |
| Val Ser Ile Gly His Pro Ser; | (SEQ ID NO: 118) |
| Thr His Ser His Arg Pro Ser; | (SEQ ID NO: 119) |
| Ile Thr Asn Pro Leu Thr Thr; | (SEQ ID NO: 120) |
| Ser Ile Gln Ala His His Ser; | (SEQ ID NO: 121) |
| Leu Asn Trp Pro Arg Val Leu; | (SEQ ID NO: 122) |
| Tyr Tyr Tyr Ala Pro Pro Pro; | (SEQ ID NO: 123) |
| Ser Leu Trp Thr Arg Leu Pro; | (SEQ ID NO: 124) |
| Asn Val Tyr His Ser Ser Leu; | (SEQ ID NO: 125) |
| Asn Ser Pro His Pro Pro Thr; | (SEQ ID NO: 126) |
| Val Pro Ala Lys Pro Arg His; | (SEQ ID NO: 127) |
| His Asn Leu His Pro Asn Arg; | (SEQ ID NO: 128) |
| Tyr Thr Thr His Arg Trp Leu; | (SEQ ID NO: 129) |
| Ala Val Thr Ala Ala Ile Val; | (SEQ ID NO: 130) |
| Thr Leu Met His Asp Arg Val; | (SEQ ID NO: 131) |
| Thr Pro Leu Lys Val Pro Tyr; | (SEQ ID NO: 132) |
| Phe Thr Asn Gln Gln Tyr His; | (SEQ ID NO: 133) |
| Ser His Val Pro Ser Met Ala; | (SEQ ID NO: 134) |
| His Thr Tbr Val Tyr Gly Ala; | (SEQ ID NO: 135) |
| Thr GIu Thr Pro Tyr Pro Thr; | (SEQ ID NO: 136) |
| Leu Thr Tbr Pro Phe Ser Ser; | (SEQ ID NO: 137) |
| Gly Val Pro Leu Thr Met Asp; | (SEQ ID NO: 138) |
| Lys Leu Pro Thr Val Leu Arg; | (SEQ ID NO: 139) |
| Cys Arg Phe His Gly Asn Arg; | (SEQ ID NO: 140) |
| Tyr Thr Arg Asp Phe Glu Ala; | (SEQ ID NO: 141) |
| Ser Ser Ala Ala Gly Pro Arg; | (SEQ ID NO: 142) |
| Ser Leu Ile Gln Tyr Ser Arg; | (SEQ ID NO: 143) |
| Asp Ala Leu Met Trp Pro Xaa; | (SEQ ID NO: 144) |
| Ser Ser Xaa Ser Leu Tyr Ile; | (SEQ ID NO: 145) |
| Phe Asn Thr Ser Thr Arg Thr; | (SEQ ID NO: 146) |
| Tbr Val Gln His Val Ala Phe; | (SEQ ID NO: 147) |
| Asp Tyr Ser Phe Pro Pro Leu; | (SEQ ID NO: 148) |
| Val Gly Ser Met Glu Ser Leu; | (SEQ ID NO: 149) |
| Phe Xaa Pro Met Ile Xaa Ser; | (SEQ ID NO: 150) |
| Ala Pro Pro Arg Val Thr Met; | (SEQ ID NO: 151) |
| Tie Ala Thr Lys Tbr Pro Lys; | (SEQ ID NO: 152) |
| Lys Pro Pro Leu Phe Gln Ile; | (SEQ ID NO: 153) |
| Tyr His Thr Ala His Asn Met; | (SEQ ID NO: 154) |
| Ser Tyr Ile Gln Ala Thr His; | (SEQ ID NO: 155) |
| Ser Ser Phe Ala Thr Phe Leu; | (SEQ ID NO: 156) |
| Thi Thr Pro Pro Asn Phe Ala; | (SEQ ID NO: 157) |
| Ile Ser Leu Asp Pro Arg Met; | (SEQ ID NO: 158) |
| Ser Leu Pro Leu Phe Gly Ala; | (SEQ ID NO: 159) |
| Asn Leu Leu Lys Thr Thr Leu; | (SEQ ID NO: 160) |
| Asp Gln Asn Leu Pro Arg Arg; | (SEQ ID NO: 161) |
| Ser His Phe Glu Gln Leu Leu; | (SEQ ID NO: 162) |
| Tbr Pro Gln Leu His His Gly; | (SEQ ID NO: 163) |
| Ala Pro Leu Asp Arg Ile Thr; | (SEQ ID NO: 164) |
| Phe Ala Pro Leu Ile Ala His; | (SEQ ID NO: 165) |
| Ser Trp Ile Gln Thr Phe Met; | (SEQ ID NO: 166) |
| Asn Thr Trp Pro His Met Tyr; | (SEQ ID NO: 167) |
| Glu Pro Leu Pro Thr Thr Leu; | (SEQ ID NO: 168) |
| His Gly Pro His Leu Phe Asn; | (SEQ ID NO: 169) |
| Tyr Leu Asn Ser Tbr Leu Ala; | (SEQ ID NO: 170) |
| His Leu His Ser Pro Ser Gly; | (SEQ ID NO: 171) |
| Tbr Leu Pro His Arg Leu Asn; | (SEQ ID NO: 172) |
| Ser Ser Pro Arg Glu Val His; | (SEQ ID NO: 173) |
| Asn Gln Vat Asp Thr Ala Arg; | (SEQ ID NO: 174) |
| Tyr Pro Thr Pro Leu Leu Thr; | (SEQ ID NO: 175) |
| His Pro Ala Ala Phe Pro Trp; | (SEQ ID NO: 176) |
| Leu Leu Pro His Ser Ser Ala; | (SEQ ID NO: 177) |
| Leu Glu Thr Tyr Thr Ala Ser; | (SEQ ID NO: 178) |
| Lys Tyr Val Pro Leu Pro Pro; | (SEQ ID NO: 179) |
| Ala Pro Leu Ala Leu His Ala; | (SEQ ID NO: 180) |
| Tyr Glu Ser Leu Leu Thr Lys; | (SEQ ID NO: 181) |
| Ser His Ala Ala Ser Gly Thr; | (SEQ ID NO: 182) |
| Gly Leu Ala Thr Val Lys Ser; | (SEQ ID NO: 183) |
| Gly Ala Thr Ser Phe Gly Leu; | (SEQ ID NO: 184) |
| Lys Pro Pro Gly Pro Val Ser; | (SEQ ID NO: 185) |
| Thr Leu Tyr Val Ser Gly Asn; | (SEQ ID NO: 186) |
| His Ala Pro Phe Lys Ser Gln; | (SEQ ID NO: 187) |
| Val Ala Phe Thr Arg Leu Pro; | (SEQ ID NO: 188) |
| Leu Pro Tbr Arg Thr Pro Ala; | (SEQ ID NO: 189) |
| Ala Ser Phe Asp Leu Leu Ile; | (SEQ ID NO: 190) |
| Arg Met Asn Thr GIu Pro Pro; | (SEQ ID NO: 191) |
| Lys Met Thr Pro Leu Thr Thr; | (SEQ ID NO: 192) |
| Ala Asn Ala Thr Pro Leu Leu; | (SEQ ID NO: 193) |
| Tbr Ile Trp Pro Pro Pro Val; | (SEQ ID NO: 194) |
| Gln Thr Lys Val Met Thr Thr; | (SEQ ID NO: 195) |
| Asn His Ala Val Phe Ala Ser; | (SEQ ID NO: 196) |
| Leu His Ala Ala Xaa Thr Ser; | (SEQ ID NO: 197) |
| Thr Trp Gln Pro Tyr Phe His; | (SEQ ID NO: 198) |
| Ala Pro Leu Ala Leu His Ala; | (SEQ ID NO: 199) |
| Thr Ala His Asp Leu Thr Val; | (SEQ ID NO: 200) |
| Asn Met Thr Asn Met Leu Thr; | (SEQ ID NO: 201) |
| Gly Ser Gly Leu Ser Gln Asp; | (SEQ ID NO: 202) |
| Thr Pro Ile Lys Tbr Ile Tyr; | (SEQ ID NO: 203) |
| Ser His Leu Tyr Arg Ser Ser; and | (SEQ ID NO: 204) |
| His Gly Gln Ala Trp Gln Phe. | (SEQ ID NO: 205) |

Xaa May be any amino acid.

For covalently linking the javelin to a melanoma antigen, it may be desirable to add, to the javelin, a “linker region” containing chemical structures which facilitate the linkage reaction. For example, where the javelin is a peptide, a linker region, preferably, but not by way of limitation, containing 1-4 amino acids may be added. As one specific, non-limiting example, where the linking reaction utilizes sulfhydrl groups, a single Cys residue, or a linker peptide such as Cys Gly Ser Gly (SEQ ID NO: 206) may be added to the amino- or carboxy-terminus of a javelin peptide.

A single melanoma antigen may be attached to one or more javelin molecules. A single javelin molecule may be attached to one or more melanoma antigens. A javelin can be attached or incorporated anywhere in an antigen, but preferably does not substantially structurally distort the melanoma specific/selective epitope. For example, in the case of peptidic antigens and one or more peptidic javelin, a javelin can be positioned at the amino terminus of the antigen, at the carboxyl terminus of the antigen, at any point within the amino acid sequence of the antigen, or at any combination of the above.

As specific nonlimiting examples, some of the javelinized melanoma antigens that can be prepared from the tyrosinase-derived melanoma antigen YMDGTMSQV (SEQ ID NO: 207) include the following Jav-peptides: HWDFAWPWYMDGTMSQV (SEQ ID NO: 208, YMDGTMSQVHWDFAWPW (SEQ ID NO: 209), HWDFAWPWYMDGTMSQVHWDFAWPW (SEQ ID NO: 210) or HWDFAWPWYMDGTMSQVWPWAFDWH (SEQ ID NO: 211), where bold face, underlined sequence denotes the javelin and non-bold face, non-underlined sequence denotes the antigenic sequence. Specific nonlimiting examples of javelinized melanoma antigens having the linker, denoted by italics, GSG include: HWDFAWPWGSGYMDGTMSQV (SEQ ID NO: 211) and HWDFAWPWGSGYMDGTMSQVGSGWPWAFDWH (SEQ ID NO:212).

Additional nonlimiting examples ofjavelinized melanoma antigens are provided in Tables 1-7 below. In each of the tables, xxx is an amino acid linker between 0 and 10 amino acids long, the bold letters represent the amino acid sequence of the javelin and the non-bold capital letters represent the amino acid sequence of the melanoma antigen. In particular, Table 1 shows nonlimiting examples ofjavelinized melanoma antigens derived from tyrosinase (consecutively, SEQ ID NOS:213-242); Tables 2 and 3 show nonlimiting examples ofjavelinized melanoma antigens derived from gp100 (consecutively, SEQ ID NOS:243-382); Table 4 shows nonlimiting examples of javelinized melanoma antigens derived from MART-1 (consecutively, SEQ ID NOS: 383-422); Table 5 shows nonlimiting examples of javelinized melanoma antigens derived from MAGE-1 (consecutively, SEQ ID NOS: 423-462); Table 6 shows nonlimiting examples ofjavelinized melanoma antigens derived from MAGE-1/3 (consecutively, SEQ ID NOS: 463-492); and Table 7 shows nonlimiting examples of javelinized melanoma antigens derived from MAGE-3 (consecutively, SEQ ID NOS: 493-502).

TABLE 1
Javelinized melanoma antigens
derived from tyrosinase
I. Derived from peptide YMDGTMSQV

HWDFAWPWxxxYMDGTMSQV
YMDGTMSQVgsgHWDFAWPW
HWDFAWPWxxxYMDGTMSQVxxxHWDFAWPW
HWDFAWPWYMDGTMSQV
YMDGTMSQVHWDFAWPW
HWDFAWPWYMDGTMSQVHWDFAWPW
YMDGTMSQVxxxWPWAFDWH
HWDFAWPWxxxYMDGTMSQVxxxWPWAFDWH
YMDGTMSQVWPWAFDWH
HWDFAWPWYMDGThISQVWPWAFDWH

II. Derived from peptide YMNGTMSQV

HWDFAWPWxxxYMNGTMSQV
YMNGTMSQVxxxHWDFAWPW
HWDFAWPWxxxYMNGTMSQVxxxHWDFAWPW
HWDFAWPWYMNGTMSQV
YMNGTMSQVHWDFAWPW
HWDFAWPWYMNGTMSQVHWDFAWPW
YMNGTMSQVxxxWPWAFDWH
HWDFAWPWxxxYMNGTMSQVxxxWPWAFDWH
YMNGTMSQVWPWAFDWH
HWDFAWPWYMNGTMSQVWPWAFDWH

III. Derived from peptide MLLAVLYCL

HWDFAWPWxxxMLLAVLYCL
MLLAVLYCLxxxHWDFAWPW
HWDFAWPWxxxMLLAVLYCLxxxHWDFAWPW
HWDFAWPWMLLAVLYCL
MLLAVLYCLHWDFAWPW
HWDFAWPWMLLAVLYGLHWDFAWPW
MLLAVLYCLxxxWPWAFDWH
HWDFAWPWxxxMLLAVLYCLxxxWPWAFDWH
MLLAVLYCLWPWAFDWH
HWDFAWPWMLLAVLYCLWPWAFDWH

TABLE 2
Javelinized melanoma antigens
derived from Gp100 (209-217)
I. Derived from peiflide IMDQVPFSV

HWDFAWPWxxxLMDQVPFSV
IMDQVPFSVxxxHWDFAWPW**
where amino acid linker xxx is gsg
HWDFAWPWxxxIMDQVPFSVxxxHWDFAWPW
HWDFAWPWIMDQVPFSV
IMDQVPFSVHWDFAWPW
HWDFAWPWIMDQVPFSVHWDFAWPW
IMDQVPFSVxxxWPWAFDWH
HWDFAWPWxxxIMDQVPFSVxxxWPWAFDWH
IMDQVPFSVWPWAFDWH
HWDFAWPWIMDQVPFSVWPWAFDWH

II. Derived from peitide ITDQVPFSV

HWDFAWPWxxxITDQVPFSV
ITDQVPFSVxxxHWDFAWPW
HWDFAWPWxxxITDQVPFSVxxxHWDFAWPW
HWDFAWPWITDQVPFSV
ITDQVPFSVHWDFAWPW
HWDFAWPWITDQVPFSVHWDFAWPW
ITDQVPFSVxxxWPWAFDWH
HWDFAWPWxxxITDQVPFSVxxxWPWAFDWH
ITDQVPFSVWPWAFDWH
HWDFAWPWITDQVPFSVWPWAFDWH

III. Derived from peptide TITDQVPFSV

HWDFAWPWxxxITDQVPFSV
TITDQVPFSVxxxHWDFAWPW
HWDFAWPWxxxTITDQVPFSVxxxHWDFAWPW
HWDFAWPWTITDQVPFSV
TITDQVPFSVHWDFAWPW
HWDFAWPWTITDQVPFSVHWDFAWPW
TITDQVPFSVxxxWPWAFDWH
HWDFAWPWxXXTITDQVPFSVxxxWPWAFDWH
TITDQVPFSVWPWAFDWH
HWDFAWPWTITDQVPFSVWPWAFDWH

TABLE 3
Javelinized melanoma antigens
derived from Gp100 (280-288)
I. Derived from peptide YLEPGVTV

HWDFAWPWxxxYLEPGVTV
YLEPGVTVxxxHWDFAWPW
HWDFAWPWxxxYLEPGVTVxxxHWDFAWPW
HWDFAWPWYLEPGVTV
YLEPGVTVHWDFAWPW
HWDFAWPWYLEPGVTVHWDFAWPW
YLEPGVTVxxxWPWAFDWH
HWDFAWPWxxxYLEPGVTVxxxWPWAFDWH
YLEPGVTVWPWAFDWH
HWDFAWPWYLEPGVTVWPWAFDWH

II. Derived from peptide YLEPGVTVA

HWDFAWPWxxxYLEPGVTVA
YLEPGVTVAxxxHWDFAWPW
HWDFAWPWYLEPGVTVAxxxHWDFAWPW
HWDFAWPWYLEPGVTVA
YLEPGWfVAHWDFAWPW
HWDFAWPWYLEPGVTVAHWDFAWPW
YLEPGVTVAxxxWPWAFDWH
HWDFAWPWxxxYLEPGVTVAxxxWPWAFDWH
YLEPGVTVAWPWAFDWH
HWDFAWPWYLEPGVTVAWPWAFDWH

III. Derived from peptide KTWGQYWQV

HWDFAWPWxxxKTWGQYWQV
KTWGQYWQVxxxHWDFAWPW
HWDFAWPWxxxKTWGQYWQVxxxHWDFAWPW
HWDFAWPWKTWGQYWQV
KTWGQYWQVHWDFAWPW
HWDFAWPWKTWGQYWQVHWDFAWPW
KTWGQYWQVxxxWPWAFDWH
HWDFAWPWxxxKTWGQYWQVxxxWPWAFDWH
KTWGQYWQVWPWAFDWH
HWDFAWPWKTWGQYWQVWPWAFDWH

IV. Derived from peptide KTWGQYWQVL

HWDFAWPWxxxKTWGQYWQVL
KTWGQYWQVLxxxHWDFAWPW
HWDFAWPWxxxKTWGQYWQVLxxxHWDFAWPW
HWDFAWPWKTWGQYWQVL
KTWGQYWQVLHWDFAWPW
HWDFAWPWKTWGQYWQVLHWDFAWPW
KTWGQYWQVLxxxWPWAFDWH
HWDFAWPWxxxKTWGQYWQVLxxxWPWAFDWH
KTWGQYWQVLWPWAFDWH
HWDFAWPWKTWGQYWQVLWPWAFDWH

V. Derived from peptide VLKRCLLHL

HWDFAWPWxxxVLKRCLLHL
VLKRCLLHLxxxHWDFAWPW
HWDFAWPWxxxVLKRCLLHLxxxHWDFAWPW
HWDFAWPWVLKRCLLHL
VLKRCLLHLHWDFAWPW
HWDFAWPWVLKRCLLHLHWDFAWPW
VLKRCLLHLxxxWPWAFDWH
HWDFAWPWxxxVLKRCLLHLxxxWPWAFDWH
VLKRCLLHLWPWAFDWH
HWDFAWPWVLKRCLLHLWPWAFDWH

VI. Derived from petide LNVSLADTN

HWDFAWPWxxxLNVSLADTN
LNVSLADTNxxxHWDFAWPW
HWDFAWPWxxxLNVSLADTNxxxHWDFAWPW
HWDFAWPWLNVSLADTN
LNVSLADTNHWDFAWPW
HWDFAWPWLNVSLADTNHWDFAWPW
LNVSLADTNxxxWPWAFDWH
HWDFAWPWxxxLNVSLADTNxxxWPWAFDWH
LNVSLADTNWPWAFDWH
HWDFAWPWLNVSLADTNWPWAFDWH

VII. Derived from peptide SLADTNSLAV

HWDFAWPWxxxSLADTNSLAV
SLADTNSLAVxxxHWDFAWPW
HWDFAWPWxxxSLADTNSLAVxxxHWDFAWPW
HWDFAWPWSLADTNSLAV
SLADTNSLAVHWDFAWPW
HWDFAWPWSLADTNSLAVxxxHWDFAWPW
SLADTNSLAVxxxWPWAFDWH
HWDFAWPWxxxSLADTNSLAVxxxWPWAFDWH
SLADTNSLAVWPWAFDWH
HWDFAWPWSLADTNSLAVWPWAFDWH

VIII. Derived from peptide LLDGTATLRL

HWDFAWPWxxxLLDGTATLRL
LLDGTATLRLXXXHWDFAWPW
HWDFAWPWxxxLLDGTATLRLxxxHWDFAWPW
HWDFAWPWLLDGTATLRL
LLDGTATLRLHWDFAWPW
HWDFAWPWLLDGTATLRLHWDFAWPW
LLDGTATLRLxxxWPWAFDWH
HWDFAWPWxxxLLDGTATLRLxxxWPWAFDWH
LLDGTATLRLWPWAFDWH
HWDFAWPWLLDGTATLRLWPWAFDWH

IX. Derived from peptide VLYRYGSFSV

HWDFAWPWxxxVLYRYGSFSV
VLYRYGSFSVxxxHWDFAWPW
HWDFAWPWxxxVLYRYGSFSVxxxHWDFAWPW
HWDFAWPWVLYRYGSFSV
VLYRYGSFSVHWDFAWPW
HWDFAWPWVLYRYGSFSVHWDFAWPW
VLYRYGSFSVxxxWPWAFDWH
HWDFAWPWxxxVLYRYGSFSVxxxWPWAFDWH
VLYRYGSFSVWPWAFDWH
HWDFAWPWVLYRYGSFSVWPWAFDWH

X. Derived from peptide ALDGGNKHFL

HWDFAWPWxxxALDGGNKHFL
ALDGGNKHFLxxxHWDFAWPW
HWDFAWPWxxxALDGGNKHFLxxxHWDFAWpW
HWDFAWPWALDGGNKHFL
ALDGGNKHFLHWDFAWPW
HWDFAWPWALDGGNKHFLHWDFAWPW
ALDGGNKHFLxxxWPWAFDWH
HWDFAWFWxxxALDGGNKHFLxxxWPWAFDWH
ALDGGNKHFLWPWAFDWH
HWDFAWPWALDGGNKHFLWPWAFDWH

XI. Derived from peptide VLPSPACOLV

HWDFAWPWxxxVLPSPACQLV
VLPSPACQLVxxxHWDFAWPW
HWDFAWPWxxxVLPSPACQLVxxxHWDFAWPW
HWDFAWPWVLPSPACQLV
VLPSPACQLVHWDFAWPW
HWDFAWPWVLPSPACQLVHWDFAWPW
VLPSPACQLVxxxWPWAFDWH
HWDFAWPWxxxVLPSPACQLVxxxWPWAFDWH
VLPSPACQLVWPWAFDWH
HWDFAWPWVLPSPACQLVWPWAFDWH

TABLE 4
Javelinized melanoma antigens
derived from MART-1/MELAN A
I. Derived from peptide AAGIGILTV

HWDFAWPWxxxAAGIGILTV
AAGIGILTVxxxHWDFAWPW
HWDFAWPWxxxAAGIGILTVxxxHWDFAWPW
HWDFAWPWAAGIGILTV
AAGIGILTVHWDFAWPW
HWDFAWPWAAGIGILTVHWDFAWPW
AAGIGILTVmWPWAFDWH
HWDFAWPWxxxAAGIGILTVxxxWPWAFDWH
AAGIGILTVWPWAFDWH
HWDFAWPWAAGIGILTVWPWAFDWH

II. Derived from peptide EAAGIGILTV

HWDFAWPWxxxEAAGIGLLTV
EAAGIGILTVxxxHWDFAWPW
HWDFAWPWxxxEAAGIGILTVxxxHWDFAWPW
HWDFAWPWEAAGIGILTV
EAAGIGILTVHWDFAWPW
HWDFAWPWEAAGIGILTVHWDFAWPW
EAAGIGILTVxxxWPWAFDWH
HWDFAWPWxxxEAAGIGILTVxxxWPWAFDWH
EAAGIGILTVWPWAFDWH
HWDFAWPWEAAGIGILTVWPWAFDWH

III. Derived from peptide EAAGOGILTVI

HWDFAWPWxxxEAAGQGILTVI
EAAGOGILTVIxxxHWDFAWPW
HWDFAWPWxxxEAAGOGILTVIxxxHWDFAWPW
HWDFAWPWEAAGOGILTVI
EAAGOGILTVIHWDFAWPW
HWDFAWPWEAAGOGILTVIHWDFAWPW
EAAGOGILTVIxxxWPWAFDWH
HWDFAWPWxxxEAAGOGILTVIxxxWPWAFDWH
EAAGOGILTVIWPWAFDWH
HWDFAWPWEAAGOGILTVIWPWAFDWH

IV. Derived from peptide ILTVILGVL

HWDFAWPWxxxILTVILGVL
ILTVILGVLxxxHWDFAWPW
HWDFAWPWxxxILTVILGVLxxxHWDFAWPW
HWDFAWPWILTVILGVL
ILTVILGVLHWDFAWPW
HWDFAWPWILTVILGVLHWDFAWPW
ILTVILGVLxxxWPWAFDWH
HWDFAWPWxxxILTVILGVLxxxWPWAFDWH
ILTVILGVLWPWAFDWH
HWDFAWPWILTVILGVLWPWAFDWH

TABLE 5
Javelinized melanoma antigens derived from MAGE-1
I. Derived from MAGE-1(15) (peptide ALEAQQEAL)

HWDFAWPWxxxALEAQQEAL
ALEAQQEALxxxHWDFAWPW
HWDFAWPWxxxALEAQQEALxxxHWDFAWPW
HWDFAWPWALEAQQEAL
ALEAQQEALHWDFAWPW
HWDFAWPWALEAQQEALHWDFAWPW
ALEAQQEALxxxWPWAFDWH
HWDFAWPWxxxALEAQQEALxxxWPWAFDWH
ALEAQQEALWPWAFDWH
HWDFAWPWALEAQQEALWPWAFDWH

II. Derived from MAGE-1 (93) (peptide ILESLFRAV)

HWDFAWPWxxxILESLFRAV
ILESLFRAVxxxHWDFAWPW
HWDFAWPWxxxILESLFRAVxxxHWDFAWPW
HWDFAWPWILESLFRAV
ILESLFRAVHWDFAWPW
HWDFAWPWILESLFRAVHWDFAWPW
ILESLFRAVxxxWPWAFDWH
HWDFAWPWxxxILESLFRAVxxxWPWAFDWH
ILESLFRAVWPWAFDWH
HWDFAWPWILESLFRAVWPWAFDWH

III. Derived from MAGE-1 (7) (peptide SLHCKPEEAL)

HWDFAWPWxxxSLHCKPEEAL
SLHCKPEEALxxxHWDFAWPW
HWDFAWPWxxxSLHCKPEEALxxxHWDFAWPW
HWDFAWPWSLHCKPEEAL
SLHCKPEEALHWDFAWPW
HWDFAWPWSLHCKPEEALHWDFAWPW
SLHCKPEEALxxxWPWAFDWH
HWDFAWPWxxxSLIIGKPEEALxxxWPWAFDWH
SLHCKPEEALWPWAFDWH
HWDFAWPWSLHCKPEEALWPWAFDWH

IV. Derived from MAGE-1 (37) peptide PLVLGTLEEV)

HWDFAWPWxxxPLVLGTLEEV
PLVLGTLEEVxxxHWDFAWPW
HWDFAWPWxxxPLVLGTLEEVxxxHWDFAWPW
HWDFAWPWPLVLGTLEEV
PLVLGTLEEVHWDFAWPW
HWDFAWPWPLVLGTLEEVHWDFAWPW
PLVLGTLEEVxxxWPWAFDWH
HWDFAWPWxxxPLVLGTLEEVxxxWPWAFDWH
PLVLGTLEEVWPWAFDWH
HWDFAWPWIPLVLGTLEEVWPWAFDWH

TABLE 6
Javelinized melan ma antigens
derived from MAGE 1/3
I. Derived from MAGE-1/3 (174) peptide CLGLSYDGL)

HWDFAWPWxxxCLGLSYDGL
CLGLSYDGLxxxHWDFAWPW
HWDFAWPWxxxCLGLSYDGLxxxHWDFAWPW
HWDFAWPWCLGLSYDGL
CLGLSYDGLHWDFAWPW
HWDFAWPWCLGLSYDGLHWDFAWPW
CLGLSYDGLxxxWPWAFDWH
HWDFAWPWxxxCLGLSYDGLxxxWPWAFDWH
CLGLSYDGLWPWAFDWH
HWDFAWPWCLGLSYDGLWPWAFDWH

II. Derived from MAGE-1/3 (174)
(peptide CLGLSYDGLL

HWDFAWPWxxxCLGLSYDGLL
CLGLSYDGLLxxxHWDFAWPW
HWDFAWPWxxxCLGLSYDGLLxxxHWDFAWPW
HWDFAWPWCLGLSYDGLL
CLGLSYDGLLHWDFAWPW
HWDFAWPWCLGLSYDGLLHWDFAWPW
CLGLSYDGLLxxxWPWAFDWH
HWDFAWPWxxxCLGLSYDGLLxxxWPWAFDWH
CLGLSYDGLLWPWAFDWH
HWDFAWPWCLGLSYDGLLWPWAFDWH

III. Derived from MAGE-1/3 (114)
(peptide LLKYRAREPV)

HWDFAWPWxxxLLKYRAREPV
LLKYRAREPVxxxHWDFAWPW
HWDFAWPWxxxLLKYRAREPVxxxHWDFAWPW
HWDFAWPWLLKYRAREPV
LLKYRAREPVHWDFAWPW
HWDFAWPWLLKYRAREPVHWDFAWPW
LLKYRAREPVxxxWPWAFDWH
HWDFAWPWxxxLLKYRAREPVxxxWPWAFDWH
LLKYRAREPVWPWAFDWH
HWDFAWPWLLKYRAREPVWPWAFDWH

TABLE 7
Javelinized melanoma antigens derived from MAGE-3

HWDFAWPWxxxFLWGPRALV
FLWGPRALVxxxHWDFAWPW
HWDFAWPWxxxFLWGPRALVxxxHWDFAWPW
HWDFAWPWFLWGPRALV
FLWGPRALVHWDFAWPW
HWDFAWPWFLWGPRALVHWDFAWPW
FLWGPRALVxxxWPWAFDWH
HWDFAWPWxxxFLWGPRALVxxxWPWAFDWH
FLWGPRALVWPWAFDWH
HWDFAWPWFLWGPRALVWPWAFDWH

Javelinization of melanoma antigens may also incorporate CpG motif sequences (see Krieg, 2000, Curr. Opinion Immunol. 12:35-43) at the C-terminal end of the javelin sequence preceding the melanoma antigen. Alternatively, CpG sequences can be javelinized separately and co-administered with hsp bound non-covalently to Jav-CpG and Jav-melanoma antigen. Immunotherapeutic compositions that may be administered to the human subject may also include, in addition to hsp bound non-covalently to Jav-melanoma antigen, a mixture of Jav-CpG sequences.

In further non-limiting embodiments, one or more T-helper peptides may be linked to the antigen of interest or separately javelinized and co-administered with javelinized melanoma antigen.

A javelin molecule may be covalently linked to a melanoma antigen using any method known in the art. In determining the method of linking to be used, particular chemical characteristics of the melanoma antigen may favor the choice of one method over another. For example, where the melanoma antigen comprises carbohydrate groups, a carbohydrate-based coupling method may advantageously be used (see below).

A pamphlet published by Pierce. Chemical Company, entitled “Double Agents™ Cross-Linking Reagents Selection Guide” (published in 1999, and available from Pierce Chemical Co. as Catalog #1600310) provides a useful set of criteria for selecting a proper agent including the following. Cross-linking reagents are identified by their acronyms, which would be recognized by the skilled artisan. Cleavable and/or non-cleavable cross-linkers may be used. If lysines and sulfhydryl groups are available for cross-linking, one may consider using a heterobifunctional amine/sulfhydryl reactive agent such as AMAS, BMPS, EMCS, sulfo-EMCS, GMBS, sulfo-GMBS, sulfo-KMUS, MBS, sulfo-MBS, SBAP, SIA, SIAB, sulfo-SIAB, SMCC, LC-SMCC, SMPB, SMPH, sulfo-SMPB, SVSB, BMPA, EMCA, KMUA, SMPT, sulfo-LC-SMPT, SPDP, LC-SPDP, and sulfo-LC-SPDP. If it is desirable to first react the agent with an —SH group on one molecule (e.g., the javelin) before coupling to an NH₂ on a second molecule (e.g., the melanoma antigen), it may be desirable, from among the aforelisted agents, to use BMPA, EMCA or KMUA. If it is desirable to incorporate a carboxyl (COOH) group into one molecule (e.g., the javelin) to facilitate coupling to the second molecule (e.g., the melanoma antigen), useful cross-linking reagents may include heterobifunctional, sequential sulfhydryl to amine-reactive agents such as BMPA, EMCA, or KMUA. If one of the components to be linked (e.g., the melanoma antigen) lacks reactive groups or if the presence or identity of such groups is unknown, it may be desirable to use a heterofunctional-photoreactive cross-linking agent such as ANB-NOS, NHS-ASA, sulfo-NHS-LC-ASA, sulfo-HSAB, SASD, sulfo-SAPB, SANPAH, sulfo-SANPAH, SFAD, ABH, EMCH, KMUH, M₂C₂H, MPBH, ASBA, sulfo-NHS-LC-ASA, SASD, and APDP. Additional information may be found in the Pierce pamphlet and/or in Hermanson, 1995, “Bioconjugate Technologies”, Academic Press, Inc., Pierce Product #20002GJ, and Wong, 1991, “Chemistry of Protein Conjugation and Cross-Linking, CRC Press, Inc., Pierce Product No. 15010GJ.

In one particular non-limiting set of embodiments, the present invention provides for covalently linking a javelin molecule containing a terminal Cys residue to a melanoma antigen comprising a terminal NH₂ group (or ajavelin molecule containing a terminal NH₂ residue to a melanoma antigen comprising a terminal Cys residue) using standard techniques, for example, using an amine-sulffiydryl cross-linker such as N-(α-maleimidodoacetoxy)-succinimide ester (“AMAS”) or “KMUS” (Pierce Chemical Co.). Such methods would generally involve reductive methylation of the javelin molecule to block N-termini, cross-linking of blocked peptide at pH 6.5-7.5 using sulfo-KMUS or AMAS, and reacting the succinimide group of the modified javelin with the melanoma antigen at pH 8-9. A detailed description of such a protocol may be found in Pierce Product Description No. 22295; May, 1989, Biochem 28:1718; and Satyre et al., 1984, J. Med. Chem. 27:1325.

In another non-limiting set of embodiments, the present invention provides for covalently linking a javelin molecule to a melanoma antigen via a photo-reactive cross linker. An example of one such cross-linker is N-5-azido-2-nitrobenzyloxy-succinimide (“ANB-NOS”).

In yet another non-limiting set of embodiments, the present invention provides for covalently linking a javelin molecule to a melanoma antigen via a method which attaches the javelin to a carbohydrate group on the melanoma antigen. Cross-linking reagents which may be used to effect such linkage include N-(E-maleimidocaproicacid)hydrazide (“EMCH”), N-(K-maleimidoundecanoic acid) hyrdazide (“KMUH”), 4-(4-N-maleimidophenyl)-butyric acid hydrazide HCl (“MPBH”), “MPBA” or photoreactive agents (see Pierce Pamphlet, cited supra).

Where one particular method of linking is appropriate to the melanoma antigen, the javelin molecule can be engineered to contain a “linker region” containing amino acid residues or other chemical structures which are appropriate to the selected linking method.

In yet further embodiments of the invention, a javelin molecule may be linked to a melanoma antigen by the creation of a fusion protein, whereby a nucleic acid encoding a melanoma antigen protein or peptide is linked, in the proper reading frame, to a javelin-encoding nucleic acid. The javelin may be introduced at either terminus. Alternatively, nucleic acid may be engineered to position one or more javelin peptide within the body of the melanoma antigen. The nucleic acid may be used to produce its encoded protein using standard techniques.

### 4.1.8. Heat Shock Proteins

The term “heat shock protein”, as used herein, refers to stress proteins (including homologs thereof expressed constitutively), including, but not limited to, gp96, hsp90, BiP, hsp70, hsp60, hsp40, hsc70, hsp170 and hsp10. Hsp target may be prepared from a natural source, expressed recombinantly, or chemically synthesized.

For example, cDNAs which may be used to express other heat shock proteins include, but are not limited to, gp96: human: Genebank Accession No. X15187; Maki et al., Proc. Natl. Acad. Sci. U.S.A. 87:5658-5562; mouse: Genebank Accession No. M16370; Srivastava et al., Proc. Natl. Acad. Sci. U.S.A. 84:3807-3811; BiP: human: Genebank Accession No. M19645, Ting et al., 1988, DNA 7:275-286; mouse Genebank Accession No. U16277, Haas et al., 1988, Proc. Natl. Acad. Sci. U.S.A. 85:2250-2254; hsp70: human: Genebank Accession No. M24743, Hunt et al., 1985, Proc. Natl. Acad. Sci. U.S.A. 82:6455-6489; mouse: Genebank Accession No. M35021, Hunt et al., 1990, Gene 87:199-204; and hsp40: human: Genebank Accession No. D49547, Ohtsuka, 1993, Biochem. Biophys. Res. Commun. 197:235-240. Such sequences may be expressed using any appropriate expression vector known in the art. Suitable vectors include, but are not limited to, herpes simplex viral based vectors such as pHSVI (Geller et al., 1990, Proc. Natl. Acad. Sci. U.S.A. 87:8950-8954); retroviral vectors such as MFG (Jaffee et al., 1993, Cancer Res. 53:2221-2226), and in particular Moloney retroviral vectors such as LN, LNSX, LNCX, and LXSN (Miller and Rosman, 1989, Biotechniques 7:980-989); vaccinia viral vectors such as MVA (Sutter and Moss, 1992, Proc. Natl. Acad. Sci. U.S.A. 89:10847-10851); adenovirus vectors such as pJM17 (Ali et al., 1994, Gene Therapy 1:367-384; Berker, 1988, Biotechniques 6:616-624; Wand and Finer, 1996, Nature Medicine 2:714-716); adeno-associated virus vectors such as AAV/neo (Mura-Cacho et al., 1992, J. Immunother. 11:231-237); pcDNA3 (InVitrogen); pET 11 a, pET3a, pET11d, pET3d, pET22d, and pET12a (Novagen); plasmid AH5 (which contains the SV40 origin and the adenovirus major late promoter); pRC/CMV (InVitrogen); pCMU II (Paabo et al., 1986, EMBO J. 5:1921-1927); pZipNeo SV (Cepko et al., 1984, Cell 37:1053-1062) and pSRα (DNAX, Palo Alto, Calif.).

A cDNA for human hsp70 may be cloned by oligonucleotide directed polymerase chain reaction (“PCR”) from a human brain cDNA library, using primers designed based on the known sequence of the hsp70 gene and considering the vector into which it is to be inserted. For example, the resulting hsp70 cDNA may be cloned into the pET27a vector (Novagen) to form expression vector pET27hhsp70, which may be propagated so that the inserted cDNA may be sequenced. The sequence of the inserted cDNA should conform to the known sequence of human hsp70. Preferably, the cDNA may encode a protein having the amino acid sequence set forth in GeneBank having an accession number AAD21816. Expression vector pET27hhsp70 may then be transfected into Escherichia coli strain HMS174(DE3) (Novagen). The genotype of this strain is F⁻ recA1 hsdR (r_{K12}⁻mK₁₂⁺) Rif^{R} (DE3). This strain is a K12 strain of E. coli. The resulting expression strain of transfected bacter may be referred to as HMS174 (DE3) hhsp70. Cells from the expression strain may be confirmed to be free of all prophage, and viability and retention of the expression construct may be confirmed. To prepare sufficient amounts of hsp70 for clinical use, 50 liters of the expression strain HMS174 (DE3) hhsp70 may be grown in animal free medium containing 122.5 g phytone peptone, 588.0 g yeast extract, 367.5 g sodium chloride, 9.8 g methionine, and ultrapure water to 50 liters. Before inoculation with expression strain bacteria, the medium may be sterilized, and supplemented with kanamycin to a final concentration of 30 micrograms per milliliter. The medium may then be inoculated with one liter of an overnight culture of the HMS174(DE3) bacteria. The pH, level of dissolved oxygen and temperature inside the fermenter reactor may be carefully monitored. When the culture reaches an optical density of approximately 1.160 (for example, approximately 2.5 hours after inoculation), IPTG (isopropyl-B-D-thiogalactopyranoside; Boehringer Mannheim Corp.) may be added to a final concentration of 1 mM to initiate the induction of expression of human hsp70. Growth may then be continued until an optical density of approximately 2.7 is attained (for example, after an additional 3.5 hours following addition of IPTG). The fermented bacteria may then be harvested by centrifugation using standard techniques. The bacterial pellet may be resuspended in one liter of buffer containing the following ingredients: 1.93 g sodium phosphate (monobasic), 5.11 g sodium phosphate (dibasic), 1.46 g sodium chloride, 0.74 g EDTA, 100 ml glycerol and 900 ml ultrapure water. The resuspended bacteria may then optionally be frozen on dry ice before further processing. To continue processing, the frozen bacteria may be thawed if necessary, and then may be disrupted by pressure lysis. The resulting lysate may then be cleared and sterile filtered (and again, optionally frozen at −80° C. prior to further processing).

For example but not by way of limitation, a bacterial lysate comprising human hsp70 may be subjected to the following three-step protocol to purify human hsp70. The specific example provided utilizes 750 liters of lysate; adjustments may be made for different lysate volumes. In the first step of the protocol, 750 ml. of the cleared lysate, prepared as set forth above, may be diluted two-fold with 20 mM sodium phosphate buffer pH 7.0. This 1500 ml of diluted and cleared bacterial lysate may be loaded onto a 1.9 liter column (13 cm×15 cm) of DEAE Sephacel (Pharmacia) which has been previously equilibrated with 2 column volumes of buffer A (20 mM sodium phosphate buffer 7.0, 25 mM sodium chloride, 10 mM ammonium sulfate, 1 mM DTT). After loading, the column may be washed with 6 column volumes of buffer A before elution is carried out with two column volumes of buffer B (20 mM sodium phosphate buffer pH 7.0, 85 mM sodium chloride, 10 mM ammonium sulfate, 1 mM DTT). Somewhat less than 4 liters of eluate (e.g. 3.8 liters) may be expected to be recovered. The eluate may then be diafiltered (buffer exchanged) against a 10-fold volume (e.g., 38 liters for 3.8 liters of eluate) of buffer C (20 mM sodium citrate pH 6.0, 85 mM sodium chloride, 10 mM ammonium sulfate, 1 mM DTT).

In the second step of the protocol, a column having one tenth the volume of eluate (e.g., 380 ml, 9 cm×6.1 cm) of Q-sepharose fast flow (Pharmacia) may be equilibrated with one column volume of buffer C (supra). The buffer exchanged eluate (e.g., 380 ml) from the DEAE column may then be run over the Q-sepharose column, and the flow through may be collected (e.g., 4.18 liters). The purpose of this column is to reduce or eliminate the endotoxin content of the hsp70 being purified.

In the third step of the protocol, a 700 ml column (13 cm×5.5 cm) of custom cGMP ATP agarose (Sigma-Aldrich Fine Chemicals) may be equilibrated in one column volume of buffer D (20 mM sodium citrate pH 6.0, 85 mM sodium chloride, 10 mM ammonium sulfate). To the eluate from the Q-sepharose column (e.g. 4.18 liters) magnesium acetate may be added to a final concentation of 0.25 mM, and the resulting solution may be run over the ATP agarose column. The column may then be washed with six column volumes of buffer D and finally eluted with 2 column volumes of buffer E (20 mM sodium citrate pH 6.0, 85 mM sodium chloride, 10 mM ammonium sulfate, 1 mM magnesium acetate, 1 mM adenosine triphosphate (ATP)). The eluted material, which may be reasonably expected to be human hsp70 having a purity of greater than 95 percent) may be buffer exchanged into phosphate buffered saline and concentrated to a final concentration of 10-20 mg/ml, and then sterile filtered.

### 4.1.9. Complexes of Javelinized Melanoma Antigens and Heat Shock Proteins

In one set of embodiments of the invention, a single javelinized melanoma antigen may be bound to a single heat shock protein or, alternatively, to a plurality of heat shock proteins. In another set of embodiments of the invention, a plurality of javelinized melanoma antigens can be bound to a single heat shock protein or, alternatively, to a plurality of heat shock proteins. Further, in nonlimiting embodiments of the invention, different javelinized melanoma antigens and different types of heat shock protein may be combined; for example, javelinized gp100 and tyrosinase derived peptides may be complexed with hsp70, or javelinized gp100 peptide may be complexed with hsp70 and gp96, or javelinized gp100 and tyrosinase peptides may be complexed with hsp70 and gp96, in the same formulation. Analogous combinations may be made using other javelinized melanoma antigens and heat shock proteins.

Complexes between javelinized melanoma antigens and heat shock proteins can be generated by a variety of methods. In one embodiment, javelinized melanoma antigen(s) can be combined with heat shock protein(s) at molar ratios from 0.01:1 to 100:1, and preferably at molar ratios from 0.1:1 to 10:1, of Jav-antigen:heat shock protein, and at weight/volume ratios of from 1:1000 to 1:1. The molecules may be combined in an aqueous solution that is buffered in the range between pH 4.5 and pH 9 and more preferably in the range pH 6 to pH 8. Examples of buffering compounds include Tris based buffer, phosphate based buffers, bicarbonate based buffers, and succinate based buffers. The concentrations of these buffering compounds range from, but are not limited to, 1 mM to 500 mM, and more preferably range from 10 mM to 200 mM.

Salts may also be added to the complex formation solution. These salts include but are not restricted to sodium chloride, potassium chloride, ammonium chloride, ammonium sulfate, magnesium chloride, magnesium acetate, potassium acetate, sodium acetate, and combinations thereof. The concentrations of these salts may fall in the ranges of, but are not limited to, 0.1 micromolar to 500 mM, and more preferably 20 mM to 200 mM.

Other compounds that may be added to the complex formation solution include adenosine 5′ diphosphate (ADP) and analogues thereof and DMSO. Such compounds may be added at concentrations ranging from, but not limited to, 0.001 mM to 500 mM, and more preferably 0.1 mM to 100 mM.

The reactants may then be incubated at a temperature ranging from, but not limited to 4° C. to 65° C., more preferably from 20° C. to 55° C. This incubation may be carried out for a time period ranging from, but not limited to 1 minute to 4 hours, more preferably from 20 minutes to 1 hour.

As one non-limiting example, heat shock protein and peptide may be introduced into a solution to produce a 1:10 molar ratio (heat shock protein:peptide) in a buffer containing 1 mM magnesium acetate (or magnesium chloride) and 1 mM ADP. The mixture may then be incubated at 25° C. for about 1 hour and subsequently centrifuged to remove any aggregates. The resulting solution may then be sterile filtered. Of note, the buffer of choice can vary depending upon the optimal conditions identified in biochemical analysis. For example, a suitable buffer may be a Tris buffer with a pH range from 7-8 containing NaCl at a concentration of 20 mM-100 mM and DMSO at a concentration of 1.0%. A complex prepared as set forth above may be frozen in a dry ice/ethanol bath and stored at −80° C.

In another non-limiting example, purified heat shock protein (e.g. the human hsp70) may be complexed with one or more javelinized melanoma antigen(s) by combining heat shock protein at a concentration of 0.25 mg/ml with either 0.25, 0.025 or 0.0025 mg/ml of thejavelinized melanoma antigen(s) in a buffer comprising 25 mM Tris (THAM), 50 mM NaCl, 5 mM MgCl₂, 1 mM ADP, brought to pH 8.0 with acetic acid.

In another non-limiting example, heat shock protein and one or more javelinized melanoma antigen may be combined as follows: 0.25 mg/ml heat shock protein may be combined with 0.25 mg/ml javelinized melanoma antigen in 25 mM Tris (Tris) pH 8.0, 50 mM NaCl, 5 mM MgCl₂, 6.7 mM Acetate, 1 mM ADP, 0.26 mM KCl, 0.518 mM Na₂HPO₄, 0.173 mM KH₂PO₄, and a final DMSO concentration of 1%.

In another specific, non-limiting embodiment of the invention, human hsp70 at a concentration of 0.25 mg/ml may be incubated with 0.25 mg/ml, 0.025 mg/ml, or 0.0025 mg/ml of Jav-tyrosinase peptide (368-377), YMDGTMSQVGSGHWDFAWPW (SEQ ID NO: 209) in a buffer comprising 25 mM Tris (THAM), 50 mM NaCl, 5 mM MgCl₂, and 1 mM ADP, with the pH brought to 8.0 with acetic acid.

In yet another specific, non-limiting embodiment of the invention, human hsp70 at a concentration of 0.25 mg/ml may be incubated with 0.25 mg/ml, 0.025 mg/ml, or 0.0025 mg/ml of Jav-gp100 peptide (209-217) IMDQVPFSVGSGHWDFAWPW (SEQ ID NO: 245) in a buffer comprising 25 mM Tris (THAM), 50 mM NaCl, 5 mM MgCl₂, and 1 mM ADP, with the pH brought to 8.0 with acetic acid.

### 4.2 Administration of the Immunotherapeutic Complexes

The heat shock protein/Jav-antigen complexes of the invention may be administered in therapeutic amounts to subjects in need of such treatment. Subjects in need of such treatment include subjects suffering from melanoma, who have previously been diagnosed with melanoma, or who are at risk for developing melanoma (for example, persons with a family history of melanoma, with sun-sensitive skin, or with sun-damaged skin). Patients having a diagnosis of melanoma in situ, AJCC Stage I, or more advanced stages of melanoma (e.g. AJCC Stage II, III, or IV) may be treated according to the invention. HLA restriction of Jav-melanoma antigen should be considered in identifying subjects suitable for treatment, as those subjects should be of the HLA type to which response is restricted.

In nonlimiting embodiments, the likelihood that a subject suffering from or having previously been diagnosed with melanoma will respond to therapy according to the invention may be evaluated by determining whether the melanoma cells of the subject carry a particular melanoma antigen. The evaluation may also be made in a subject suspected of suffering from melanoma. Such an evaluation may be made, for example, by RT-PCR analysis as described in Berking et al., 1999, Arch. Dermatol. Res. 291(9):479-484 and Riker et al., 2000, Int. J. Cancer 86(6):818-826, which report that tumor markers for melanoma could be detected in the peripheral blood of melanoma patients.

Heat shock protein/Jav-Antigen is administered in an amount effective in inducing or maintaining a therapeutic immune response. A “therapeutic immune response” refers to an increase in humoral and/or cellular immunity directed toward melanoma cells and which is minimally or non-cross reactive with non-malignant cells, of a magnitude sufficient to protect against the development, growth and/or spread of melanoma cells in a patient. Preferably, but not by way of limitation, the induced level of immunity (humoral or cellular) directed toward the melanoma antigen is at least four fold, and preferably at least 16-fold greater than the levels of immunity directed toward the antigen prior to the administration of the immunotherapeutic composition.

The level of immunity may be measured by any method known in the art. The immune response may be measured qualitatively in vivo, wherein an arrest in progression or a remission of melanoma in the subject is considered to indicate the induction of a therapeutic immune response. As another example of a means for determining, in vivo, whether a therapeutic immune response has occurred, skin tests for delayed hypersensitivity responses may be performed, which may, e.g. utilize 0.05 ml volumes containing 10 μg melanoma antigen. Tests may be applied intradermally at suitable time points in the treatment, for example, prior to the first vaccination and after the final immunizations and are read at 48 hours. The presence of a response is a positive indication that a therapeutic immune response toward the javelinized melanoma antigens is being induced in the subject.

Alternatively, the immune response may be evaluated by laboratory tests. as one non-limiting example, humoral immunity may be evaluated by measuring antibody titers. Other examples of suitable tests include ELISPOT assays, proliferation assays, and/or cytokine release assays, which may be performed as described in Lewis et al., 1999, Int. J. Cancer 87(3:391-398), which is incorporated by reference. Tetramer assays may also or alternatively be used to measure specifically reactive T cells against melanoma antigen, as described in Jaeger, E. et al., 1996, Int. J. Cancer 66: 162-169. An additional test which may be used singly or in combination with the above is the chromium release assay, as described in Wu, J. Y. et al., 1992, J. Immunol. 148: 1519-1525, in which a panel of HLA typed melanoma cell lines are used. Many of the cell lines have been studied extensively for expression of a variety of tyrosinase and gp100 antigens. For example, SK MEL 29 which is HLA A2+, expresses both the tyrosinase and gp100 antigens. Lymphocytes from the patient from whom SK MEL 29 was derived recognize tyrosinase strongly and can serve as a positive control. (Nestle et al., 1998, Nature Medicine 4: 328-332; Rosenberg et al., 1998, Nature Medicine 4:321-327).

In particular nonlimiting embodiments of the invention, the immunotherapeutic composition comprises, for each javelinized melanoma antigen, between 1 to 100 μg of the javelinized melanoma antigen and 100 μg of heat shock protein, such as hsp70, delivered in a volume of 0.4 ml of a 25 mM Tris (THAM) buffer containing 50 mM NaCl, 5 mM MgCl₂, 6.7 mM acetate, 1 mM ADP, 0.26 mM KCl, 0.518 mM Na₂HPO₄, 1 percent DMSO and has a pH of 8.00.

In one group of specific, non-limiting embodiments of the invention, a complex between human hsp70 and Jav-tyrosinase peptide(368-377) may be prepared as set forth in the preceding section and administered such that a patient receives 100 ricrograms of hsp70 with 1, 10 or 100 micrograms of Jav-tyrosinase peptide in a volume of 0.4 ml of buffer containing 50 mM NaCl, 5 mM MgCl₂, 6.7 mM acetate, 1 mM ADP, 0.26 mM KCl, 0.518 mM Na₂HPO₄, 1 percent DMSO and has apH of 8.00.

In a second group of specific, non-limiting embodiments of the invention, a complex between human hsp70 and Jav-gp100 peptide(209-217) may be prepared as set forth in the preceding section and administered such that a patient receives 100 micrograms of hsp70 with 1, 10 or 100 micrograms of Jav-gp100 peptide in a volume of 0.4 ml of buffer containing 50 mM NaCl, 5 mM MgCl₂, 6.7 mM acetate, 1 mM ADP, 0.26 mM KCl, 0.518 mM Na₂HPO₄, 1 percent DMSO and has a pH of 8.00.

In a third group of specific, non-limiting embodiments of the invention, a complexes between human hsp70 and Jav-tyrosinase peptide(368-377) and between human hsp70 and Jav-gp100 peptide (209-217) may be prepared as set forth in the preceding section and administered such that a patient receives 200 micrograms of hsp70 with 1, 10 or 100 micrograms of Jav-tyrosinase peptide and of Jav-gp100 peptide in a volume of 0.8 ml of buffer containing 50 mM NaCl, 5 mM MgCl₂, 6.7 mM acetate, 1 mM ADP, 0.26 mM KCl, 0.518 mM Na₂HPO₄, 1 percent DMSO and has a pH of 8.00.

An immunotherapeutic composition according to the invention may be initially a freeze-dried or lyophilized preparation of complex to which buffer solution is added. Alternatively, the immunotherapeutic composition may be delivered as a dry powder formulation. Further formulations, such as sustained release preparations, microsphere or liposome based preparations, etc. may also be used, which are prepared using techniques that are known in the art.

Immunization may be delivered in an inpatient or outpatient setting. Preferably, a plurality of vaccinations are provided at regular time intervals to optimize and sustain the immune response. In one preferred embodiment, a human subject may receive a total of at least 5 vaccinations. With regard to the time interval between vaccinations, the immunotherapeutic composition may administered at weekly intervals, at bi-weekly intervals, or a combination of both. As another example, the immunotherapeutic composition may be administered at weeks 0, 1, 2, 6, and 18. In another example, the second and third vaccinations may be administered up to one week, and the fourth and fifth vaccinations, can be administered up to two weeks, earlier or later.

Immunization can be carried out by any route known in the medical art, including, but not limited to, intradermal, subcutaneous, intraperitoneal, intrathecal, intravenous, or intramuscular injection, or mucosal, intratracheal, or oral administration. In one preferred specific embodiment, the immunotherapeutic composition may be administered subcutaneously in a volume of 0.8 ml normal saline The sites of immunization can be the same or be varied. For example, the vaccine may be administered on the arm, leg, or belly, or on any combination thereof. For example, the first administration may be on the right arm, second administration the left arm, the third administration on the right thigh, the fourth administration on the left thigh, the fifth on the right side of the belly and the sixth on the left side of the belly.

The present invention further provides for administration of additional biologically active agents either concurrently with or before or after administration of the immunotherapeutic hsp/Jav-peptide composition. Examples of suitable biologically active agents include but are not limited to cytokines, such as interleukin-2 (IL-2), granulocyte/macrophage colony stimulating factor (GM-CSF), interferon α2b, interferon γ, and/or a lymphokine.

In another set of nonlimiting embodiments, the present invention provides for methods comprising contacting patient-derived antigen presenting cells with javelinized melanoma antigens bound to heat shock proteins in vitro, followed by re-administration of the antigen presenting cells into the patient. For example, approximately 2-3×10⁶ dendritic cells prepared from 50 mls of peripheral blood using “Lymphoprep” (GIBCO BRL, Baithersburg, Md.) may be “loaded” with heat shock protein/Jav-antigen prior to administration to a patient. The administration of the antigen presenting cells sensitized with the immunotherapeutic composition may occur before, concurrent with or after the administration to the patient of the javelizined antigens bound to the heat shock, proteins or other molecular chaperones at the defined weekly intervals. The antigen presenting cells can be autologous or heterologous immune cells such as cytotoxic T-lymphocytes, B-lymphocytes, and preferably macrophages and more preferably dendritic cells.

In a patient treated according to the invention, it is desirable to monitor the patient for autoimmune or hypersensitivity reactions to components of the vaccine. Such reactions are theoretical possibilities because melanocyte differentiation antigens such as tyrosinase and gp100 are present in the melanosomes of pigmented cells in the skin, retina, and uvea. Induction of immunity against these antigens might therefore induce destruction of pigmented cells, resulting in vitiligo. It is also possible that choroiditits could be induced. However, in patients immunized with gp100 or tyrosinase peptides in trials conducted at MSKCC, National Cancer Institute (NCI) and the University of Pittsburgh, there have been no reported cases of treatment related retinopathy as detected by slit-lamp examinations. Nevertheless, it may be desirable to monitor patients by slit-lamp ophthalmic examination.

While the invention has been described in connection with what is presently considered to be the most practical and preferred embodiment, it will be apparent to one skilled in the art that various changes and modifications can be made therein. Thus, the present invention is not to be limited to the disclosed embodiments, but on the contrary, is intended to cover various modifications and equivalent arrangements included within the spirit and scope of the appended claims.

Various references are cited herein, the contents of which are hereby incorporated by reference in their entireties

We claim:

1. A method of inducing an immune response in a subject comprising administering to said subject a therapeutic amount of an immunotherapeutic composition comprising a heat shock protein and a melanoma antigen, wherein the melanoma antigen is selected from the group consisting of tyrosinase, tyrosinase related protein 1, tyrosinase related protein 2, gp100, MAGE antigens, BAGE antigens, NYES01, MART antigens, GM2, antigenic portions thereof and combinations thereof, wherein the melanoma antigen is covalently bound to a javelin molecule, and wherein the melanoma antigen bound to the javelin molecule is non-covalently bound to the heat shock protein.

2. The method of claim 1 wherein the melanoma antigen is a peptide selected from the group consisting of peptides having sequences Tyr-Met-Asp-Gly-Thr-Met-Ser-Gln-Val (SEQ ID NO: ), Tyr-Met-Asn-Gly-Thr-Met-Ser-Gln-Val (SEQ ID NO: ), Met-Leu-Leu-Ala-Val-Leu-Tyr-Val-Leu (SEQ ID NO: ), Met-Ser-Leu-Gln-Arg-Gln-Phe-Leu-Arg (SEQ ID NO: ), Leu-Leu-Gly-Pro-Gly-Arg-Pro-Tyr-Arg (SEQ ID NO: ), Val-Met-Gly-Thr-Leu-Val-Ala-Leu-Val (SEQ ID NO ), Leu-Leu-Ala-Val-Leu-Tyr-Cys-Leu (SEQ ID NO: ), Ala-Ala-Gly-Ile-Gly-Ile-Leu-Thr-Val (SEQ ID NO: ), Glu-Ala-Ala-Gly-Ile-Gly-Ile-Leu-Thr-Val (SEQ ID NO: ), Ala-Ala-Gly-Ile-Gly-Ile-Leu-Thr-Val (SEQ ID NO: ), Ile-Leu-Thr-Val-Ile-Leu-Gly-Val-Leu (SEQ ID NO: ), Ile-Met-Asp-Gln-Val-Pro-Phe-Ser-Val (SEQ ID NO: ), Ile-Thr-Asp-Gln-Val-Pro-Phe-Ser-Val (SEQ ID NO: ); Thr-Ile-Thr-Asp-Gln-Val-Pro-Phe-Ser-Val (SEQ ID NO: ), Tyr-Leu-Glu-Pro-Gly-Val-Thr-Val (SEQ ID NO: ), Tyr-Leu-Glu-Pro-Gly-Val-Thr-Val-Ala (SEQ ID NO: ), Lys-Thr-Trp-Gly-Gln-Tyr-Trp-Gln-Val (SEQ ID NO: ), Lys-Thr-Trp-Gly-Gln-Tyr-Trp-Gln-Val-Leu (SEQ ID NO: ), Val-Leu-Lys-Arg-Cys-Leu-Leu-His-Leu (SEQ ID NO: ), Leu-Asn-Val-Ser-Leu-Ala-Asp-Thr-Asn (SEQ ID NO: ), Ser-Leu-Ala-Asp-Thr-Asn-Ser-Leu-Ala-Val (SEQ ID NO: ), Leu-Leu-Asp-Gly-Thr-Ala-Thr-Leu-Arg-Leu (SEQ ID NO; ), Val-Leu-Tyr-Arg-Tyr-Gly-Ser-Phe-Ser-Val (SEQ ID NO: ), Ala-Leu-Asp-Gly-Gly-Asn-Lys-His-Phe-Leu (SEQ ID NO: ), Val-Leu-Pro-Ser-Pro-Ala-Cys-Gln-Leu-Val (SEQ ID NO:
), Ala-Leu-Glu-Ala-GIn-Gln-Glu-Ala-Leu (SEQ ID NO: ), Ile-Leu-Glu-Ser-Leu-Phe-Arg-Ala-Val (SEQ ID NO: ), Ser-Leu-His-Cys-Lys-Pro-Glu-Glu-Ala-Leu (SEQ ID NO: ), Pro-Leu-Val-Leu-Gly-Thr-Leu-Glu-Glu-Val (SEQ ID NO: ), Cys-Leu-Gly-Leu-Ser-Tyr-Asp-Gly-Leu (SEQ ID NO: ), Cys-Leu-Gly-Leu-Ser-Tyr-Asp-Gly-Leu-Leu (SEQ ID NO: ), Leu-Leu-Lys-Tyr-Arg-Ala-Arg-Glu-Pro-Val (SEQ ID NO: ), Phe-Leu-Trp-Gly-Pro-Arg-Ala-Leu-Val (SEQ ID NO: ), Glu-Ala-Asp-Pro-Thr-Gly-His-Ser-Tyr (SEQ ID NO: ), Ser-Leu-Asp-Asp-Tyr-Asn-His-Leu-Val (SEQ ID NO: ), Thr-Leu-Asp-Ser-Gln-Val-Met-Ser-Leu (SEQ ID NO: ), Val-Met-Gly-Thr-Leu-Val-Ala-Leu-Val (SEQ ID NO: ) and epitope-containing fragments thereof.

3. The method of claim 1 wherein the heat shock protein is selected from the group consisting of hsp70, hsp90, gp96, BiP, hsp40, hsp170 and mixtures thereof.

4. The method of claim 2 wherein the heat shock protein is selected from the group consisting of hsp70, hsp90, gp96, BiP, hsp40, hsp170 and mixtures thereof.

5. The method of claim 1 wherein the melanoma antigen is covalently joined to one or more javelin molecule selected from the group consisting of peptides having the sequences His-Trp-Asp-Phe-Ala-Trp-Pro-Trp (SEQ ID NO: ), Trp-Pro-Trp-Ala-Phe-Asp-Trp-His (SEQ ID NO: ) and combinations thereof.

6. The method of claim 5 wherein the javelin molecule is joined to the melanoma antigen by a peptide linker.

7. The method of claim 2 wherein the melanoma antigen is covalently joined to one or more javelin molecule selected from the group consisting of peptides having the sequences His-Trp-Asp-Phe-Ala-Trp-Pro-Trp (SEQ ID NO: ), Trp-Pro-Trp-Ala-Phe-Asp-Trp-His (SEQ ID NO: ) and combinations thereof.

8. The method of claim 7 wherein the javelin molecule is joined to the melanoma antigen by a peptide linker.

9. The method of claim 3 wherein the melanoma antigen is covalently joined to one or more javelin molecule selected from the group consisting of peptides having the sequences His-Trp-Asp-Phe-Ala-Trp-Pro-Trp (SEQ ID NO: ), Trp-Pro-Trp-Ala-Phe-Asp-Trp-His (SEQ ID NO: ) and combinations thereof.

10. The method of claim 9 wherein the javelin molecule is joined to the melanoma antigen by a peptide linker.

11. The method of claim 4 wherein the melanoma antigen is covalently joined to one or more javelin molecule selected from the group consisting of peptides having the sequences His-Trp-Asp-Phe-Ala-Trp-Pro-Trp (SEQ ID NO: ), Trp-Pro-Trp-Ala-Phe-Asp-Trp-His (SEQ ID NO: ) and combinations thereof.

12. The method of claim 11 wherein the javelin molecule is joined to the melanoma antigen by a peptide linker.

13. The method of claim 1 wherein the melanoma antigen is covalently joined to one or more javelin molecule selected from the group consisting of peptides having the sequences Phe-Trp-Gly-Leu-Trp-Pro-Trp-Glu (SEQ ID NO: ), Glu-Trp-Pro-Trp-Leu-Gly-Trp-Phe (SEQ ID NO: ) and combinations thereof.

14. The method of claim 13 wherein the javelin molecule is joined to the melanoma antigen by a peptide linker.

15. The method of claim 2 wherein the melanoma antigen is covalently joined to one or more javelin molecule selected from the group consisting of peptides having the sequences Phe-Trp-Gly-Leu-Trp-Pro-Trp-Glu (SEQ ID NO: ), Glu-Trp-Pro-Trp-Leu-Gly-Trp-Phe (SEQ ID NO: ) and combinations thereof.

16. The method of claim 15 wherein the javelin molecule is joined to the melanoma antigen by a peptide linker.

17. The method of claim 3 wherein the melanoma antigen is covalently joined to one or more javelin molecule selected from the group consisting of peptides having the sequences Phe-Trp-Gly-Leu-Trp-Pro-Trp-Glu (SEQ ID NO: ), Glu-Trp-Pro-Trp-Leu-Gly-Trp-Phe (SEQ ID NO: ) and combinations thereof.

18. The method of claim 17 wherein the javelin molecule is joined to the melanoma antigen by a peptide linker.

19. The method of claim 4 wherein the melanoma antigen is covalently joined to one or more javelin molecule selected from the group consisting of peptides having the sequences Phe-Trp-Gly-Leu-Trp-Pro-Trp-Glu (SEQ ID NO: ), Glu-Trp-Pro-Trp-Leu-Gly-Trp-Phe (SEQ ID NO: ) and combinations thereof.

20. The method of claim 19 wherein the javelin molecule is joined to the melanoma antigen by a peptide linker.

21. The method of claim 1 wherein the immunotherapeutic compositions comprises (a) a first melanoma antigen comprising a peptide having the sequence Tyr-Met-Asp-Gly-Thr-Met-Ser-Gln-Val (SEQ ID NO: ) covalently linked to a first javelin molecule and (b) a second melanoma antigen comprising a peptide having the sequence Ile-Met-Asp-Gln-Val-Pro-Phe-Ser-Val (SEQ ID NO: ) linked to a second javelin molecule.

22. The method of claim 21 wherein the first and second javelin molecules are the same and comprise a peptide having the sequence His-Trp-Asp-Phe-Ala-Trp-Pro-Trp (SEQ ID NO: ) and wherein the heat shock protein is hsp70.

23. An immunotherapeutic composition comprising a heat shock protein and a melanoma antigen, wherein the melanoma antigen is selected from the group consisting of tyrosinase, tyrosinase related protein 1, tyrosinase related protein 2, gp100, MAGE antigens, BAGE antigens, NYES01, MART antigens, GM2, antigenic portions thereof and combinations thereof, wherein the melanoma antigen is covalently bound to a javelin molecule, and wherein the melanoma antigen bound to the javelin molecule is non-covalently bound to the heat shock protein.

24. The immunotherapeutic composition of claim 23 wherein the melanoma antigen is selected from the group consisting of peptides having the sequences Tyr-Met-Asp-Gly-Thr-Met-Ser-Gln-Val (SEQ ID NO: ), Tyr-Met-Asn-Gly-Thr-Met-Ser-Gln-Val (SEQ ID NO: ), Met-Leu-Leu-Ala-Val-Leu-Tyr-Val-Leu (SEQ ID NO: ), Met-Ser-Leu-Gln-Arg-Gln-Phe-Leu-Arg (SEQ ID NO: ), Leu-Leu-Gly-Pro-Gly-Arg-Pro-Tyr-Arg (SEQ ID NO: ), Val-Met-Gly-Thr-Leu-Val-Ala-Leu-Val (SEQ ID NO ), Leu-Leu-Ala-Val-Leu-Tyr-Cys-Leu (SEQ ID NO: ), Ala-Ala-Gly-Ile-Gly-Ile-Leu-Thr-Val (SEQ ID NO: ), Glu-Ala-Ala-Gly-Ile-Gly-Ile-Leu-Thr-Val (SEQ ID NO: ), Ala-Ala-Gly-Ile Gly-Ile-Leu-Thr-Val (SEQ ID NO: ), Ile-Leu-Thr-Val-Ile-Leu-Gly-Val-Leu (SEQ ID NO: ), Ile-Met-Asp-Gln-Val-Pro-Phe-Ser-Val (SEQ ID NO: ), Ile-Thr-Asp-Gln-Val-Pro-Phe-Ser-Val (SEQ ID NO: ), Thr-Ile-Thr-Asp-Gln-Val-Pro-Phe-Ser-Val (SEQ ID NO: ), Tyr-Leu-Glu-Pro-Gly-Val-Thr-Val (SEQ ID NO: ), Tyr-Leu-Glu-Pro-Gly-Val-Thr-Val-Ala (SEQ ID NO: ), Lys-Thr-Trp-Gly-Gln-Tyr-Trp-Gln-Val (SEQ ID NO: ), Lys-Thr-Trp-Gly-Gin-Tyr-Trp-Gln-Val-Leu (SEQ ID NO: ), Val-Leu-Lys-Arg-Cys-Leu-Leu-His-Leu (SEQ ID NO: ), Leu-Asn-Val-Ser-Leu-Ala-Asp-Thr-Asn (SEQ ID NO: ), Ser-Leu-Ala-Asp-Thr-Asn-Ser-Leu-Ala-Val (SEQ ID NO: ), Leu-Leu-Asp-Gly-Thr-Ala-Thr-Leu-Arg-Leu (SEQ ID NO; ), Val-Leu-Tyr-Arg-Tyr-Gly-Ser-Phe-Ser-Val (SEQ ID NO: ), Ala-Leu-Asp-Gly-Gly-Asn-Lys-His-Phe-Leu (SEQ ID NO: ), Val-Leu-Pro-Ser-Pro-Ala-Cys-Gln-Leu-Val (SEQ ID NO: ), Ala-Leu-Glu-Ala-Gln-Gln-Glu-Ala-Leu (SEQ ID NO: ), Ile-Leu-Glu-Ser-Leu-Phe-Arg-Ala-Val (SEQ ID NO: ), Ser-Leu-His-Cys-Lys-Pro-Glu-Glu-Ala-Leu (SEQ ID NO: ), Pro-Leu-Val-Leu-Gly-Thr-Leu-Glu-Glu-Val (SEQ ID NO: ), Cys-Leu-Gly-Leu-Ser-Tyr-Asp-Gly-Leu (SEQ ID NO: ), Cys-Leu-Gly-Leu-Ser-Tyr-Asp-Gly-Leu-Leu (SEQ ID NO: ), Leu-Leu-Lys-Tyr-Arg-Ala-Arg-Glu-Pro-Val (SEQ ID NO: ), Phe-Leu-Trp-Gly-Pro-Arg-Ala-Leu-Val (SEQ ID NO: ), Glu-Ala-Asp-Pro-Thr-Gly-His-Ser-Tyr (SEQ ID NO: ), Ser-Leu-Asp-Asp-Tyr-Asn-His-Leu-Val (SEQ ID NO: ), Thr-Leu-Asp-Ser-Gln-Val-Met-Ser-Leu (SEQ ID NO: ) and Val-Met-Gly-Thr-Leu-Val-Ala-Leu-Val (SEQ ID NO: ) and epitope-containing fragments thereof.

25. The immunotherapeutic composition of claim 23 wherein the heat shock protein is selected from the group consisting of hsp70, hsp90, gp96, BiP, hsp40, hsp 170 and mixtures thereof.

26. The immunotherapeutic composition of claim 24 wherein the heat shock protein is selected from the group consisting of hsp70, hsp90, gp96, BiP, hsp40, hsp 170 and mixtures thereof.

27. The immunotherapeutic composition of claim 23 wherein the melanoma antigen is covalently joined to one or more javelin molecule selected from the group consisting of peptides having the sequences His-Trp-Asp-Phe-Ala-Trp-Pro-Trp (SEQ ID NO: ______), Trp-Pro-Trp-Ala-Phe-Asp-Trp-His (SEQ ID NO: ______ and combinations thereof.

28. The immunotherapeutic composition of claim 27 wherein the javelin molecule is joined to the melanoma antigen by a peptide linker.

29. The immunotherapeutic composition of claim 24 wherein the melanoma antigen is covalently joined to one or more javelin molecule selected from the group consisting of peptides having the sequences His-Trp-Asp-Phe-Ala-Trp-Pro-Trp (SEQ ID NO: ______), Trp-Pro-Trp-Ala-Phe-Asp-Trp-His (SEQ ID NO: ______) and combinations thereof.

30. The immunotherapeutic composition of claim 29 wherein the javelin molecule is joined to the melanoma antigen by a peptide linker.

31. The immunotherapeutic composition of claim 25 wherein the melanoma antigen is covalently joined to one or more javelin molecule selected from the group consisting of peptides having the sequences His-Trp-Asp-Phe-Ala-Trp-Pro-Trp (SEQ ID NO: ______), Trp-Pro-Trp-Ala-Phe-Asp-Trp-His (SEQ ID NO: ______) and combinations thereof.

32. The immunotherapeutic composition of claim 31 wherein the javelin molecule is joined to the melanoma antigen by a peptide linker.

33. The immunotherapeutic composition of claim 26 wherein the melanoma antigen is covalently joined to one or more javelin molecule selected from the group consisting of peptides having the sequences His-Trp-Asp-Phe-Ala-Trp-Pro-Trp (SEQ ID NO: ______), Trp-Pro-Trp-Ala-Phe-Asp-Trp-His (SEQ ID NO: ______) and combinations thereof.

34. The immunotherapeutic composition of claim 33 wherein the javelin molecule is joined to the melanoma antigen by a peptide linker.

35. The immunotherapeutic composition of claim 23 wherein the melanoma antigen is covalently joined to one or more javelin molecule selected from the group consisting of peptides having the sequences Phe-Trp-Gly-Leu-Trp-Pro-Trp-Glu. (SEQ ID NO: ______), Glu-Trp-Pro-Trp-Leu-Gly-Trp-Phe (SEQ ID NO: ______) and combinations thereof.

36. The immunotherapeutic composition of claim 35 wherein the javelin molecule is joined to the melanoma antigen by a peptide linker.

37. The immunotherapeutic composition of claim 24 wherein the melanoma antigen is covalently joined to one or more javelin molecule selected from the group consisting of peptides having the sequences Phe-Trp-Gly-Leu-Trp-Pro-Trp-Glu (SEQ ID NO: ______), Glu-Trp-Pro-Trp-Leu-Gly-Trp-Phe (SEQ ID NO: ______) and combinations thereof.

38. The immunotherapeutic composition of claim 37 wherein the javelin molecule is joined to the melanoma antigen by a peptide linker.

39. The immunotherapeutic composition of claim 25 wherein the melanoma antigen is covalently joined to one or more javelin molecule selected from the group consisting of peptides having the sequences Phe-Trp-Gly-Leu-Trp-Pro-Trp-Glu (SEQ ID NO: ______), Glu-Trp-Pro-Trp-Leu-Gly-Trp-Phe (SEQ ID NO: ______) and combinations Thereof.

40. The immunotherapeutic composition of claim 39 wherein the javelin molecule is joined to the melanoma antigen by a peptide linker.

41. The immunotherapeutic composition of claim 26 wherein the melanoma antigen is covalently joined to one or more javelin molecule selected from the group consisting of peptides having the sequences Phe-Trp-Gly-Leu-Trp-Pro-Trp-Glu (SEQ ID NO: ______), Glu-Trp-Pro-Trp-Leu-Gly-Trp-Phe (SEQ ID NO: ______) and combinations thereof.

42. The immunotherapeutic composition of claim 41 wherein the javelin molecule is joined to the melanoma antigen by a peptide linker.

43. The immunotherapeutic composition of claim 23 comprising a first and a second melanoma antigen, wherein the first melanoma antigen comprises a peptide having the sequence Tyr-Met-Asp-Gly-Thr-Met-Ser-Gln-Val (SEQ ID NO: ) and is linked to a javelin molecule, and the composition further comprises a second melanoma antigen linked to a javelin molecule.

44. The immunotherapeutic composition of claim 43 wherein the second melanoma antigen comprises a peptide having the sequence Ile-Met-Asp-Gln-Val-Pro-Phe-Ser-Val.

44. The immunotherapeutic composition of claim 43 wherein the first and second melanoma antigens are linked to the same species of javelin molecule, which comprises a peptide having the sequence His-Trp-Asp-Phe-Ala-Trp-Pro-Trp (SEQ ID NO: ) and wherein the heat shock protein is hsp70.
